SEQUENCE LISTING (20 sequences)

SEQ ID NO: 1 - DNA, 573 bp, Medicago sp.
  source 1..573: mol_type = other DNA; note = Medicago falcata
  CDS 1..573: gene = MfERF026; protein_id = 2; translation = MNSSTIEQPHNSETKSSSNSSPPPPPSQSLQIKGIRDTSKHPVY RGVRMRNWGKWVSEIREPKKKSRIWLGTFPTPEMAARAHDVAALSIKGSAAILNFPEL VNLLPRPASLAPRDIQAAATKAAHMEFPSSTASYELTEIIELPRLGNVGDFGKEFVFM DSIDTSWMFQPPCLQTMEDAIWSDIYNNYS
atgaatagtagtactattgaacaacctcataactcagaaaccaagagtagctcaaattcatcaccaccaccaccaccatcacaatccctacaaataaaaggcataagagacacaagcaagcatccagtataccgtggtgtccgaatgcgaaattggggaaaatgggtgtccgaaattcgtgagcctaagaaaaaatcccgaatatggctcggcacatttcccacaccagaaatggcagctcgagcacacgatgtagctgctcttagtataaaaggaagcgccgccattctcaacttccctgagttagtaaacttgcttcctcgtccggcttcactcgctccccgtgatattcaagcagccgctaccaaagccgctcacatggagtttccatcttcaacagcttcatatgaattgactgagataattgagcttcctcgtttgggaaacgttggagattttggaaaggagtttgtatttatggattccattgatacttcgtggatgtttcagcctccttgcttgcaaaccatggaagatgcgatttggagtgacatttataataactatagctag

SEQ ID NO: 2 - protein, 190 aa, Medicago sp.
  source 1..190: note = protein encoded by MfERF026; note = Medicago falcata
MNSSTIEQPHNSETKSSSNSSPPPPPSQSLQIKGIRDTSKHPVYRGVRMRNWGKWVSEIREPKKKSRIWLGTFPTPEMAARAHDVAALSIKGSAAILNFPELVNLLPRPASLAPRDIQAAATKAAHMEFPSSTASYELTEIIELPRLGNVGDFGKEFVFMDSIDTSWMFQPPCLQTMEDAIWSDIYNNYS

SEQ ID NO: 3 - DNA, 20 bp, synthetic construct
  source 1..20: mol_type = other DNA; note = Primer MfERFO26-F
tttatttctctccttcccat

SEQ ID NO: 4 - DNA, 21 bp, synthetic construct
  source 1..21: mol_type = other DNA; note = Primer MfERFO26-R
aaacacactaagacattcaca

SEQ ID NO: 5 - DNA, 20 bp, synthetic construct
  source 1..20: mol_type = other DNA; note = Primer MfERF026DL-F
attcaagcagccgctaccaa

SEQ ID NO: 6 - DNA, 20 bp, synthetic construct
  source 1..20: mol_type = other DNA; note = Primer MfERF026DL-R
cctttccaaaatctccaacg

SEQ ID NO: 7 - DNA, 41 bp, synthetic construct
  source 1..41: mol_type = other DNA; note = Primer MfERF026 PBE-F
gactctagagcagtcgacgatgaatagtagtactattgaac

SEQ ID NO: 8 - DNA, 41 bp, synthetic construct
  source 1..41: mol_type = other DNA; note = Primer MfERF026 PBE-R
ggcgaccggtggatcccggctatagttattataaatgtcac

SEQ ID NO: 9 - DNA, 30 bp, synthetic construct
  source 1..30: mol_type = other DNA; note = Primer MfERF026a-F
cgggatccatgaatagtagtactattgaac

SEQ ID NO: 10 - DNA, 38 bp, synthetic construct
  source 1..38: mol_type = other DNA; note = Primer MfERF026a-R
ataagaatgcggccgcctagctatagttattataaatg

SEQ ID NO: 11 - DNA, 36 bp, synthetic construct
  source 1..36: mol_type = other DNA; note = Primer MfERF026b-R
ataagaatgcggccgcaagcaagtttactaactcag

SEQ ID NO: 12 - DNA, 25 bp, synthetic construct
  source 1..25: mol_type = other DNA; note = Primer MfERF026c-F
cgggatccgtataccgtggtgtccg

SEQ ID NO: 13 - DNA, 26 bp, synthetic construct
  source 1..26: mol_type = other DNA; note = Primer MfERF026e-F
cgggatcccctcgtccggcttcactc

SEQ ID NO: 14 - DNA, 50 bp, synthetic construct
  source 1..50: mol_type = other DNA; note = Primer MfERF026 RNAi-F
cgggatccttcagaggtgaagacacacgtagatgtagctgctcttagtat

SEQ ID NO: 15 - DNA, 30 bp, synthetic construct
  source 1..30: mol_type = other DNA; note = Primer MfERF026 RNAi-R
gctctagactagctatagttattataaatg

SEQ ID NO: 16 - DNA, 24 bp, synthetic construct
  source 1..24: mol_type = other DNA; note = Primer 3xFlag-R
ctacttatcgtcatcgtccttgta

SEQ ID NO: 17 - DNA, 362 bp, synthetic construct
  source 1..362: mol_type = other DNA; note = Base fragment for reducing MfERF026 expression
cgggatccttcagaggtgaagacacacgtagatgtagctgctcttagtataaaaggaagcgccgccattctcaacttccctgagttagtaaacttgcttcctcgtccggcttcactcgctccccgtgatattcaagcagccgctaccaaagccgctcacatggagtttccatcttcaacagcttcatatgaattgactgagataattgagcttcctcgtttgggaaacgttggagattttggaaaggagtttgtatttatggattccattgatacttcgtggatgtttcagcctccttgcttgcaaaccatggaagatgcgatttggagtgacatttataataactatagctagtctagagc

SEQ ID NO: 18 - RNA, 22 nt, synthetic construct
  source 1..22: mol_type = other RNA; note = tasiRNA
ttcagaggtgaagacacacgta

SEQ ID NO: 19 - DNA, 21 bp, synthetic construct
  source 1..21: mol_type = other DNA; note = Primer Actin-F
gctgataggatgagcaaggag

SEQ ID NO: 20 - DNA, 25 bp, synthetic construct
  source 1..25: mol_type = other DNA; note = Primer Actin-R
gagcctccaatccagcagacactat

(19) United States
(12) Patent Application Publication
(10) Pub. No.: US 2025/0027100 A1
(43) Pub. Date: Jan. 23, 2025

(54) USE OF MfERF026 GENE REGULATION IN GROWTH, DEVELOPMENT, AND STRESS TOLERANCE OF MEDICAGO SATIVA

(71) Applicant: Inner Mongolia University, Hohhot City (CN)

(72) Inventors: Yiding NIU, Hohhot City (CN); Jiabin FU, Hohhot City (CN); Jinfeng HAO, Hohhot City (CN); Yu WANG, Hohhot City (CN)

(21) Appl. No.: 18/651,582

(22) Filed: Apr. 30, 2024

(30) Foreign Application Priority Data
Jul. 21, 2023 (CN) 202310897060.7

(51) Int. Cl. C12N 15/82

(52) U.S. Cl. CPC C12N 15/8218; C12N 15/8205; C12N 15/8262; C12N 2310/14

(57) ABSTRACT

Provided is use of MfERF026 regulation in growth, development, and stress tolerance of Medicago sativa, which belongs to the technical field of plant genetic engineering. Provided is use of a regulatory agent for MfERF026, which is an ethylene responsive factor (ERF) in Medicago falcata, in promoting growth and development and improving drought tolerance and cold tolerance of M. sativa. The inhibited expression of MfERF026, the ERF in M. falcata may enhance the drought tolerance and cold tolerance of the M. sativa. Moreover, the inhibited expression of MfERF026, the ERF in M. falcata, may regulate the growth and development of M. sativa, thereby accelerating the growth rate of M. sativa to increase the number of leaves. The inhibited expression of MfERF026 provides a novel theoretical basis and a candidate gene for cultivating novel leguminous forage varieties with high resistance, yield, and protein content.

### CROSS REFERENCE TO RELATED APPLICATION

This patent application claims the benefit and priority of Chinese Patent Application No. 202310897060.7 filed with the China National Intellectual Property Administration on Jul. 21, 2023, the disclosure of which is incorporated by reference herein in its entirety as part of the present application.

### REFERENCE TO SEQUENCE LISTING

A computer readable XML file entitled “GWP20231209446_seqlist”, that was created on Jun. 25, 2024, with a file size of about 24,355 bytes, contains the sequence listing for this application, has been filed with this application, and is hereby incorporated by reference in its entirety.

### TECHNICAL FIELD

The present disclosure belongs to the technical field of plant genetic engineering, and specifically relates to use of MfERF026 regulation in growth, development, and stress tolerance of Medicago sativa.

### BACKGROUND

Transcription factors (TFs), also known as trans-acting factors, are DNA-binding proteins that can specifically interact with cis-acting elements in eukaryotic gene promoter regions. The transcription of target genes is activated or inhibited through interactions of the TFs with the cis-acting elements and with other related proteins. In recent years, a series of transcription factors have been isolated from higher plants for regulating the expression of genes related to drought, low temperature, high salt concentration, hormones, pathogen response, and development. APETALA2/ethylene response element-binding proteins (AP2/EREBPs) are a large family of TFs that are widely present in plants, and their members play important regulatory roles in growth and development, organ construction, adversity stress, and hormone signal response of the plants.

Medicago sativa L. is one of the most widely planted artificial pastures in China due to high protein content and rich variety of vitamins, and prepared feed based on same is loved by various livestock because of excellent taste. Therefore, the cultivation of M. sativa exhibits a long-term significance for the sustainable development of economy, environment, and agriculture. However, long-term drought conditions generally cause adverse effects on the growth of M. sativa, thereby reducing the survival rate and affecting the yield of M. sativa.

There is currently a lack of technology for improving novel M. sativa varieties. Improving the drought resistance and increasing the growth rate of M. sativa through technological means such as molecular genetics and gene editing may greatly promote the application of M. sativa in production. However, there is a lack of technology on achieving the drought tolerance in M. sativa.

### SUMMARY

In view of the deficiencies in the prior art, an objective of the present disclosure is to provide use of a regulatory agent for MfERF026, an ethylene responsive factor (ERF) of Medicago falcata, in promoting growth and development and improving drought tolerance and cold tolerance of M. sativa.

The objective of the present disclosure is achieved by the following technical solutions:

The present disclosure provides use of a regulatory agent for MfERF026, an ERF of M. falcata, in one or more of promoting growth and development and improving drought tolerance and cold tolerance of M. sativa, where MfERF026 encodes a protein including any one of the following A1 to A4:
A1, a protein with the amino acid sequence of SEQ ID NO: 2;
A2, a protein that is obtained by substituting and/or deleting and/or adding one or more amino acid residues of A1 and has a same function as that of A1;
A3, a protein that has more than 80% identity with the amino acid sequence defined by A1 or A2 and has a same function as that of A1 or A2; and
A4, a fusion protein obtained by ligating a protein tag to an N-terminal and/or a C-terminal on any one of the proteins defined in A1 to A3.

In some embodiments, MfERF026 has the nucleotide sequence of SEQ ID NO: 1.

In some embodiments, the regulatory agent includes an agent that silences or reduces expression of MfERF026.

In some embodiments, the agent that reduces the expression of MfERF026 includes an interfering RNA for the expression of MfERF026.

The present disclosure further provides a trans-acting small interfering RNA (tasiRNA), where the tasiRNA has the nucleotide sequence of SEQ ID NO: 18.

The present disclosure further provides a base fragment for reducing MfERF026 expression obtained based on the tasiRNA, where the base fragment has the nucleotide sequence of SEQ ID NO: 17.

The present disclosure further provides a primer pair for amplifying the base fragment, where the primer pair includes an upstream primer MfERF026 RNAi-F and a downstream primer MfERF026 RNAi-R; the upstream primer MfERF026 RNAi-F has the nucleotide sequence of SEQ ID NO: 14; and the downstream primer MfERF026 RNAi-R has the nucleotide sequence of SEQ ID NO: 15.

The present disclosure further provides an expression vector for reducing MfERF026 expression, where the expression vector includes the base fragment.

The present disclosure further provides an engineering bacterium constructed based on the expression vector.

The present disclosure provides a method for constructing a transgenic M. sativa, including the following steps:
infecting a M. sativa leaf with the engineering bacterium to obtain a transformed M. sativa leaf; and
subjecting the transformed M. sativa leaf to tissue culture to obtain a transgenic M. sativa with inhibited MfERF026 expression.

The embodiments of the present disclosure has following beneficial effects:

The present disclosure provides use of a regulatory agent for MfERF026, an ERF of M. falcata, in one or more of promoting growth and development and improving drought tolerance and cold tolerance of M. sativa, where MfERF026 encodes a protein including any one of the following A1 to A4: A1, a protein with the amino acid sequence of SEQ ID NO: 2; A2, a protein that is obtained by substituting and/or deleting and/or adding one or more amino acid residues of A1 and has a same function as that of A1; A3, a protein that has more than 80% identity with the amino acid sequence defined by A1 or A2 and has a same function as that of A1 or A2; and A4, a fusion protein obtained by ligating a protein tag to an N-terminal and/or a C-terminal on any one of the proteins defined in A1 to A3.

In the present disclosure, the expression vector that inhibits the expression of MfERF026 is integrated into a M. sativa genome using transgenic technology to initially change development, drought resistance, and cold tolerance of the M. sativa.

In the present disclosure, the inhibited expression of MfERF026, an ERF of M. falcata, in the M. sativa may enhance the drought tolerance of the M. sativa. Inhibiting the expression of MfERF026 may improve the survival rate of M. sativa under drought conditions, reduce the degree of cell membrane damage of M. sativa under drought conditions to be maintained at normal levels, thereby improving the drought resistance of M. sativa. The inhibited expression of MfERF026, an ERF of M. falcata, in the M. sativa may enhance the cold tolerance of the M. sativa. Inhibiting the expression of MfERF026 may improve the survival rate of M. sativa at −7° C. to −8° C. and reduce damages to the cell membrane of M. sativa at −7° C. to −8° C., thus improving the cold tolerance of M. sativa. The inhibited expression of the ERF gene of M. falcata in M. sativa may promote the growth and development of M. sativa, thereby accelerating the growth and/or increasing the number of M. sativa leaves. Inhibiting the expression of MfERF026 in M. sativa may regulate the growth of M. sativa. Inhibiting the expression of MfERF026 in M. sativa may accelerate the growth of M. sativa and increase the number of M. sativa leaves. In summary, the inhibited expression of MfERF026 increases plant heights, number of tillers, and number of three-trifoliate leaves of M. sativa, enhances drought tolerance and cold tolerance of M. sativa, and provides a novel theoretical basis and a candidate gene for cultivating novel leguminous forage varieties with high resistance, yield, and protein content.

### BRIEF DESCRIPTION OF THE DRAWINGS

To illustrate the examples of the present disclosure or the technical solutions in the prior art more clearly, the accompanying drawings required in the examples will be briefly described below.

FIGS. 1A-IC show the relative expression of MfERF026 in M. falcata under cold stress conditions;

FIGS. 2A-2C show the relative expression of MfERF026 in M. falcata under salt stress conditions;

FIGS. 3A-3C show the relative expression of MfERF026 in M. falcata under drought stress conditions;

FIGS. 4A-4C show the relative expression of MfERF026 in M. falcata under abscisic acid (ABA) stress conditions;

FIG. 5 shows the observation results under a laser confocal microscope in the subcellular localization experiment of MfERF026 protein;

FIGS. 6A-6B show the culture results of yeast transformants using fusion expression vectors of 5 different target fragments of MfERF026 in a transcription activation experiment on different media;

FIGS. 7A-7G show the verification results of a MfERF026 transgenic line;

FIG. 8 shows the growth of transgenic lines #26 and #29 and wild-type M. sativa;

FIGS. 9A-9D show the statistical results of plant height, number of tillers, number of trifoliate leaves, and number of segments of wild-type plants and transgenic plants #26 and #29;

FIGS. 10A-10C show the statistical results of leaflet weight, leaflet area, and leaflet specific leaf weight of wild-type plants and transgenic plants #26 and #29;

FIGS. 11A-11C show the statistical results of leaflet length, leaflet width, and leaflet aspect ratio of wild-type plants and transgenic plants #26 and #29;

FIG. 12 shows the growth status of wild-type plants and transgenic plants before the drought tolerance test;

FIG. 13 shows the growth status of wild-type plants and transgenic plants under drought stress for 3 weeks;

FIG. 14 shows the growth status of wild-type plants and transgenic plants after rewatering for one week;

FIG. 15 shows the statistical results of survival rates of the drought tolerance test;

FIG. 16 shows the statistical results of the electrical conductivity before and after drought stress;

FIGS. 17A-17F show the measurement results of physiological and biochemical indicators of drought stress for MfERF026;

FIG. 18 shows the growth of plants during a cold tolerance test of the wild-type and transgenic lines;

FIG. 19 shows the survival rates of the cold tolerance test of the wild-type and transgenic lines; and

FIG. 20 shows the detection results of the electrical conductivity of the cold tolerance test of the wild-type and transgenic lines.

### DETAILED DESCRIPTION OF THE EMBODIMENTS

The present disclosure provides use of a regulatory agent for MfERF026, an ERF of M. falcata, in one or more of promoting growth and development and improving drought tolerance and cold tolerance of M. sativa, where MfERF026 encodes a protein including any one of the following A1 to A4:
A1, a protein with the amino acid sequence of SEQ ID NO: 2;
A2, a protein that is obtained by substituting and/or deleting and/or adding one or more amino acid residues of A1 and has a same function as that of A1;
A3, a protein that has more than 80% identity with the amino acid sequence defined by A1 or A2 and has a same function as that of A1 or A2; and
A4, a fusion protein obtained by ligating a protein tag to an N-terminal and/or a C-terminal on any one of the proteins defined in A1 to A3.

In the present disclosure, a protein encoded by MfERF026 has the amino acid sequence of SEQ ID NO: 2, specifically as follows:

MNSSTIEQPHNSETKSSSNSSPPPPPSQSLQIKGIRDTSKHPVYRGVRM
RNWGKWVSEIREPKKKSRIWLGTFPTPEMAARAHDVAALSIKGSAAILN
FPELVNLLPRPASLAPRDIQAAATKAAHMEFPSSTASYELTEIIELPRL
GNVGDFGKEFVFMDSIDTSWMFQPPCLQTMEDAIWSDIYNNYS.

In the present disclosure, an MfERF026 protein is located in the nucleus; and a C-terminal of the MfERF026 protein has a transcription activation activity.

In the present disclosure, MfERF026 has the nucleotide sequence of SEQ ID NO: 1, specifically as follows:

5′-ATGAATAGTAGTACTATTGAACAACCTCATAACTCAGAAACCAAGA
GTAGCTCAAATTCATCACCACCACCACCACCATCACAATCCCTACAAAT
AAAAGGCATAAGAGACACAAGCAAGCATCCAGTATACCGTGGTGTCCGA
ATGCGAAATTGGGGAAAATGGGTGTCCGAAATTCGTGAGCCTAAGAAAA
AATCCCGAATATGGCTCGGCACATTTCCCACACCAGAAATGGCAGCTCG
AGCACACGATGTAGCTGCTCTTAGTATAAAAGGAAGCGCCGCCATTCTC
AACTTCCCTGAGTTAGTAAACTTGCTTCCTCGTCCGGCTTCACTCGCTC
CCCGTGATATTCAAGCAGCCGCTACCAAAGCCGCTCACATGGAGTTTCC
ATCTTCAACAGCTTCATATGAATTGACTGAGATAATTGAGCTTCCTCGT
TTGGGAAACGTTGGAGATTTTGGAAAGGAGTTTGTATTTATGGATTCCA
TTGATACTTCGTGGATGTTTCAGCCTCCTTGCTTGCAAACCATGGAAGA
TGCGATTTGGAGTGACATTTATAATAACTATAGCTAG-3′.

In some embodiments, the regulatory agent includes an agent that silences or reduces expression of MfERF026. The agent that reduces the expression of MfERF026 includes preferably an interfering RNA for the expression of MfERF026.

In some embodiments, a process of improving the drought tolerance of M. sativa includes: improving the survival rate of M. sativa under drought conditions and/or reducing the degree of cell membrane damage of M. sativa under drought conditions. The drought tolerance of M. sativa can be improved by reducing the expression of MfERF026, thus improving the survival rate of M. sativa under drought conditions. This can keep the electrical conductivity of M. sativa at a low level under drought conditions and reduce the degree of cell membrane damage of M. sativa under drought conditions.

In some embodiments, a process of improving the cold tolerance of M. sativa includes: improving the survival rate of M. sativa under cold conditions and/or reducing the degree of cell membrane damage of M. sativa under cold conditions. Reducing the expression of MfERF026 can improve the survival rate of M. sativa at −7° C. to −8° C. and reduce damages to the cell membrane of M. sativa at −7° C. to −8° C., thus improving the cold tolerance of M. sativa.

In some embodiments, a process of promoting the growth and development of M. sativa includes: promoting the growth of M. sativa and/or increasing the number of M. sativa leaves. The growth of M. sativa can be promoted by reducing the expression of MfERF026, thereby increasing the plant height, number of tillers, number of segments, and number of trifoliate leaves of M. sativa.

The present disclosure further provides a tasiRNA, where the tasiRNA has the nucleotide sequence of SEQ ID NO: 18: 5′-TTCAGAGGTGAAGACACACGTA-3′.

The present disclosure further provides use of the tasiRNA in inhibiting MfERF026 expression. The tasiRNA can degrade the mRNA of MfERF026, blocking the formation of the MfERF026 protein to complete the inhibited expression of MfERF026. In some embodiments, the tasiRNA is ligated to a target gene fragment and introduced into M. sativa to inhibit expression of the target gene. In some embodiments, the tasiRNA is ligated before the 5′-end of the target gene fragment; and the target gene fragment is a sequence of about 300 bp on the 3′-end of the target gene. In addition to reducing the expression of MfERF026, the tasiRNA can also interfere with the expression of other genes.

The present disclosure further provides a base fragment for reducing MfERF026 expression obtained based on the tasiRNA, where the base fragment preferably has the nucleotide sequence of SEQ ID NO: 17. The base fragment has an efficiency of 87% to 99% in inhibiting the expression of MfERF026, showing an obvious inhibitory effect on the expression.

The present disclosure further provides a primer pair for amplifying the base fragment. In some embodiments, the primer pair includes an upstream primer MfERF026 RNAi-F and a downstream primer MfERF026 RNAi-R; the nucleotide sequence of the upstream primer MfERF026 RNAi-F is set forth in SEQ ID NO: 14, specifically: 5′-CGGGATCCTTCAGAGGTGAAGACACACGTAGATGTAGCTGCTCTTAGTAT-3′; and the nucleotide sequence of the downstream primer MfERF026 RNAi-R is set forth in SEQ ID NO: 15, specifically: 5′-GCTCTAGACTAGCTATAGTTATTATAAATG-3′. In some embodiments, the base fragment includes restriction endonuclease sites BamHI and XbaI to facilitate ligation with the expression vector.

In some embodiments, the upstream primer MfERF026 RNAi-F carries the tasiRNA. The base fragment of SEQ ID NO: 17 is preferably obtained M through the MfERF026 RNAi-F and MfERF026 RNAi-R, with the cDNA of M. falcata as a template. In some embodiments, the base fragment is a fragment of MfERF026 carrying tasiRNA. The base fragment can reduce the expression of MfERF026.

The present disclosure further provides a preparation for reducing MfERF026 expression, including one or more of an expression cassette, a vector, or a transgenic cell of the base fragment according to the present disclosure.

The present disclosure further provides an expression vector for reducing MfERF026 expression, where the expression vector includes the base fragment. In some embodiments, the expression vector includes a pCAM1307 plasmid.

The present disclosure further provides an engineering bacterium constructed based on the expression vector for reducing MfERF026 expression. In some embodiments, the engineering bacterium includes Agrobacterium EHA105.

The present disclosure provides a method for constructing a transgenic M. sativa, including the following steps:
infecting a M. sativa leaf with the engineering bacterium to obtain a transformed M. sativa leaf; and
subjecting the transformed M. sativa leaf to tissue culture to obtain the transgenic M. sativa with inhibited MfERF026 expression.

In some embodiments, a M. sativa leaf is infected with the engineering bacterium to obtain a transformed M. sativa leaf.

In some embodiments, a process of obtaining the engineering bacterium includes: transforming the expression vector that inhibits MfERF026 expression into the engineering bacterium, and screening a positive engineering bacterium to obtain a target engineering bacterium.

In some embodiments, the expression vector for inhibiting MfERF026 expression includes a recombinant plasmid pCAM1307-RNAi-MfERF026. In some embodiments, the engineering bacterium includes Agrobacterium EHA105. There is no particular limitation on a transformation method for transforming Agrobacterium with the expression vector, and any conventional transformation method in this field can be used. In some embodiments, the transformation method includes liquid nitrogen quick freezing and heat shock; the liquid nitrogen quick freezing is conducted for preferably 2 min; and the heat shock is conducted for preferably 30 s. After the transformation is completed, a positive colony is preferably selected to obtain positive Agrobacterium. A process of selecting the positive colony preferably includes sequentially conducting primary culture and solid culture on resulting transformed Agrobacterium. The primary culture is preferably conducted on a YEB medium at 28° C. and 190 rpm for 4 h. After the primary culture is completed, the Agrobacterium is preferably subjected to the solid culture. The solid culture is preferably conducted on a YEB solid medium containing kanamycin and rifampicin; the YEB solid medium has the kanamycin of preferably 50 mg/L and the rifampicin of 75 mg/L by mass concentration. The solid culture is preferably conducted at 28° C. for 2 d. The solid culture is preferably conducted by inverted culture. After the solid culture is completed, a colony obtained by screening is a positive colony; and the positive colony is the positive engineering bacterium, referred to as the engineering bacterium for short.

In the present disclosure, the M. sativa leaf is infected with the positive engineering bacterium to obtain the transformed M. sativa leaf.

In some embodiments, after the positive engineering bacterium is obtained, its positive bacterial colonies are subjected to expanded culture. The expanded culture is preferably conducted in a YEB medium. The expanded culture is conducted at preferably 28° C. In some embodiments, an Agrobacterium bacterial solution with an OD₆₀₀ value of 0.6 to 0.8 is obtained through the expanded culture. After the Agrobacterium bacterial solution is obtained, the bacterial solution is preferably centrifuged, and a resulting supernatant is discarded to collect positive Agrobacterium bacterial cells. The centrifugation is conducted at preferably 2,400 rpm for 15 min. After the positive Agrobacterium bacterial cells are obtained, the cells are preferably resuspended to obtain an Agrobacterium infection solution. The Agrobacterium bacterial cells are preferably resuspended in a SH3α liquid medium containing 100 μmol/L acetosyringone.

In some embodiments, M. sativa leaves are infected with the Agrobacterium infection solution to obtain transformed M. sativa leaves. The M. sativa leaves are preferably leaves of Zhongmu No. 1, more preferably leaves in well growth condition after being cultivated for 4 to 6 weeks. The M. sativa is preferably transformed by an EHA105-mediated leaf disc method. Before the transformation, the M. sativa leaves are preferably washed and sterilized in sequence to obtain pretreated M. sativa leaves. The washing is preferably conducted using ultrapure water. After the washing is completed, the sterilization is preferably conducted. The sterilization is preferably conducted using a mixed solution of hypochloric acid rinsing solution and Tween-20. In some embodiments, hypochloric acid in the hypochloric acid rinsing solution has a mass percentage of 7%. The Tween-20 and the hypochloric acid rinsing solution are at a volume ratio of preferably 1:3000. The sterilization is conducted for preferably 10 min to 13 min. After the pretreated M. sativa leaves are obtained, the pretreated M. sativa leaves and the Agrobacterium infection solution are preferably mixed to obtain a M. sativa infection system. The M. sativa infestation system is preferably vacuum infiltrated. The vacuum infiltration is preferably conducted at 0.08 MPa to 0.09 MPa for 10 min. After the vacuum infiltration is completed, the Agrobacterium suspension is preferably discarded, and the Agrobacterium on the leaf surface is removed by sucking on sterilized filter paper to obtain transformed M. sativa leaves.

In some embodiments, the transformed M. sativa leaf is subjected to tissue culture to obtain the transgenic M. sativa with inhibited MfERF026 expression.

In some embodiments, the transformed M. sativa is dark-cultured on a common medium to obtain a primary culture explant. The dark culture is preferably conducted at 24° C. for 24 h to 30 h. In some embodiments, the primary culture explant is transferred to a selection medium for subculture to obtain a callus. The subculture is conducted at preferably 24° C. for 5 to 6 weeks; and the subculture is preferably conducted once every 2 weeks. The subculture is preferably conducted until the callus is formed. The subculture is preferably completed in the dark.

In some embodiments, the callus is transferred to an MSBK medium for embryoid culture. The embryoid culture is conducted for preferably 10 d to 14 d. The embryoid culture is preferably conducted until a green embryoid appears; and the embryoid culture is preferably conducted under a light intensity of 150 μmol/m²/s at 20° C. to 24° C., with a light-to-dark ratio of 16 h: 8 h. After the embryoid culture is completed, the callus with green embryoid is obtained. In some embodiments, the callus of the green embryoid is cultured for shoot differentiation. The shoot differentiation is preferably conducted in an SH9 medium. The shoot differentiation is conducted under alight intensity of preferably 150 μmol/m²/s at preferably 20° C. to 24° C. with a light-to-dark ratio of 16 h: 8 h for 6 to 8 weeks; and during the shoot differentiation, subculture is preferably conducted once every 3 to 4 weeks. When the shoot differentiation is completed, a trifoliate plant with 2 to 3 fully-expanded leaves is obtained, which is referred to as a trifoliate plant. In some embodiments, the trifoliate plant is subjected to rooting culture to obtain rooted seedlings. The rooting culture is preferably conducted in an MSO medium. The rooting culture is preferably conducted under a light intensity of 150 μmol/m²/s at 20° C. to 24° C. with a light-to-dark ratio of 16 h: 8 h for 1 month. If vigorously growing seedlings are difficult to take root in one month, indole 3-acetic acid (IAA) is preferably added to the MSO medium to promote rooting. The IAA is added at preferably 1 mg/L.

In some embodiments, the rooted seedlings are subjected to acclimatization. The acclimatization is preferably conducted in a soil matrix including vermiculite and perlite at a volume ratio of 1:1. The acclimatization is preferably conducted under a light intensity of 150 μmol/m²/s at 20° C. to 24° C. with a light-to-dark ratio of 16 h: 8 h for 1 to 2 weeks. After the acclimatization is completed, the seedlings are preferably transplanted to a matrix containing nutrient soil and vermiculite at a volume ratio of 1:1 for culture, to obtain the transgenic M. sativa with inhibited MfERF026 expression.

In some embodiments, the expression vector that inhibits MfERF026 is integrated into the genome of the transgenic M. sativa to initially change the development, drought resistance, and cold resistance of the M. sativa in an orientation manner. The inhibited expression of MfERF026 increases plant heights, number of tillers and segments, and number of trifoliate leaves of M. sativa, enhances drought tolerance and cold tolerance of M. sativa, and provides a novel theoretical basis and a candidate gene for cultivating novel leguminous forage varieties with high anti, yield, and protein content.

In order to further illustrate the present disclosure, the technical solutions provided by the present disclosure are described in detail below in connection with accompanying drawings and examples, but these examples should not be construed to limit the claimed scope of the present disclosure.

In the present disclosure, the media used are as follows:

The minimal medium includes the following components: MgSO₄·7H₂O 185 mg/L, KNO₃ 2,830 mg/L, (NH₄)₂SO₄·463 mg/L, CaCl₂·2H₂O 166 mg/L, KH₂PO₄·400 mg/L, MnSO₄H₂O 10 mg/L, H₃BO₃ 5.0 mg/L, ZnSO₄·7H₂O 1.0 mg/L, KI 1.0 mg/L, Na₂MoO₄·2H₂O 0.1 mg/L, CuSO₄∜5H₂O 0.2 mg/L, CoCl₂·6H₂O 0.1 mg/L, EDTA-FeNa 140 mg/L, thiamine hydrochloride 5.0 mg/L, pyridoxine hydrochloride 5.0 mg/L, nicotinic acid 5.0 mg/L, and inositol 100 mg/L; and the minimal medium has a pH value of 5.8 to 6.0.

In addition to the minimal medium, the co-culture medium further consists of: sucrose 30 g/L, acetosyringone 100 μmol/L, 2,4-D 4 mg/L, 6-BA 0.5 mg/L, and phytagel 3 g/L.

In addition to the minimal medium, the callus medium (subculture medium) further consists of: sucrose 30 g/L, 2,4-D 4 mg/L, 6-BA 0.5 mg/L, timentin 200 mg/L, hygromycin B 10 mg/L, and phytagel 3 g/L.

The embryoid induction medium (MSBK medium) uses MS medium as the minimal medium and further consists of: sucrose 30 g/L, kinetin 1 mg/L, 6-BA 0.5 mg/L, timentin 150 mg/L, hygromycin B 5 mg/L, and phytagel 3 g/L, pH=5.8-6.0.

In addition to the minimal medium, the embryoid differentiation medium (SH9 medium) further consists of: sucrose 20 g/L, timentin 150 mg/L, hygromycin B 5 mg/L, and phytagel 3 g/L, pH=5.8-6.0.

The rooting medium uses MS medium as the minimal medium and further consists of: 10 g/L sucrose and 3 g/L phytagel, pH=5.8-6.0.

SH3α medium includes the following components: N6 macroelement 100 mL; SH trace salt 1 mL; SH vitamin 1 mL; inositol 100 mg; sucrose 30 g; 10 mg/mL 2,4-D 0.4 mL; 1 mg/mL 6-BA 0.5 mL; 100 mM acetosyringone (AS) 1 mL; 400 mg/mL TMT 0.5 mL; 50 mg/mL hygromycin (Hyg) 200 μL; phytagel 3 g, adding water to 1 L; pH=5.8.

N6 macroelement: MgSO₄·7H₂O 1.85 g/L, KNO₃ 28.3 g/L, (NH₄)₂SO₄·4.63 g/L, CaCl₂·2H₂O 2.475 g/L, and KH₂PO₄·4 g/L.

SH trace salt: MnSO₄H₂O 1 g/L, H₃BO₃ 0.5 g, ZnSO₄·7H₂O 0.1 g, KI 0.1 g, Na₂MoO₄·2H₂O 0.01 g, and H₂O 100 mL.

SH vitamin: nicotinic acid 0.5 g, thiamine hydrochloride B1 0.5 g, pyridoxine hydrochloride B6 0.5 g, and H₂O 100 mL.

### Example 1

Obtaining the MfERF026 Transcription Factor from M. falcata

The total RNA was extracted from the stem tissue of wild-type M. falcata plants treated with 250 mmol/L NaCl stress for 24 h.

The total RNA was extracted using the liquid nitrogen grinding method and Trizol RNA extraction reagent from Takara Biotech.

cDNA synthesis: the cDNA was synthesized by reverse transcription according to the instructions of Takara Biotech's reverse transcription kit (6210A).

Real-time quantitative polymerase chain reaction (RT-qPCR) was conducted using MfERF026-F and MfERF026-R as primers according to the instructions of PrimeSTAR® GXL DNA Polymerase Kit (TAKARA, Code No. R050A), with primer information shown in Table 1.

TABLE 1
Primers for amplifying MfERF026

| Primer name | Primer ID | 5′-3′ |
|---|---|---|
| MfERFO26-F | SEQ ID NO: 3 | TTTATTTCTCTCCTTCCCAT |
| MfERFO26-R | SEQ ID NO: 4 | AAACACACTAAGACATTCACA |

Polymerase chain reaction (PCR) conditions included: initial denaturation at 95° C. for 5 min; denaturation at 98° C. for 10 s; annealing at 51° C. for 30 s; extension at 68° C. for 1 min, 30 cycles; post-extension at 68° C. for 10 min; and storage at 4° C.

After the PCR was completed, a target fragment (about 700 bp) was recovered using a SanPrep column DNA gel recovery kit of Sangon Biotech Co., Ltd., and the operations were done according to the instructions of the kit.

The recovered target fragment was sequenced, and the sequencing results were compared to obtain the coding sequence (CDS) of MfERF026 of M. falcata. The CDS of MfERF026 had the nucleotide sequence of SEQ ID NO: 1 and the amino acid sequence of SEQ ID NO: 2.

### Example 2

Analysis of Expression Pattern of MfERF026 n M. falcata

The plant material was wild-type M. falcata that had grown normally for 30 d.

4° C. cold stress treatment: the M. falcata was placed in a 4° C. plant low-temperature incubator to allow stress treatment for 0 h, 2 h, 4 h, 8 h, 24 h, and 48 h separately.

Salt stress treatment: the M. falcata was placed in a 250 mM NaCl solution to allow stress treatment for 0 h, 2 h, 4 h, 8 h, 24 h, and 48 h separately.

Drought stress treatment: the M. falcata was placed in a 100 mM mannitol solution to allow stress treatment for 0 h, 2 h, 4 h, 8 h, 24 h, and 48 h separately.

ABA stress treatment: the M. falcata was placed in a 100 μM ABA solution to allow stress treatment for 0 h, 2 h, 4 h, 8 h, 24 h, and 48 h separately.

The root, stem, and leaf tissues of M. falcata were separated at different stress time points, wrapped in tin foil, labeled (stress conditions, stress time, and tissue type), snap-frozen in liquid nitrogen and stored at −80° C.

RNA was extracted from M. falcata under the above stress treatment conditions, and complementary DNA (cDNA) was obtained through reverse transcription. RT-qPCR was conducted using the cDNA as a template and MfERF026 DL-F and MfERF026 DL-R as primers according to the instructions of a TAKARA Biotechnology Fluorescence Quantitative Kit (RR047Q). At the same time, a glyceraldehyde-3-phosphate dehydrogenase (GAPDH) gene (GenBank accession number: GQ398120) was used as a reference gene for the quantitative polymerase chain reaction (Q-PCR). The primer sequences of MfERF026 DL-F, MfERF026 DL-R, and reference gene are shown in Table

TABLE 2
Primer information

| Primer name | Primer ID | 5′-3′ |
|---|---|---|
| MfERF026DL-F | SEQ ID NO: 5 | ATTCAAGCAGCCGCTACCAA |
| MfERF026DL-R | SEQ ID NO: 6 | CCTTTCCAAAATCTCCAACG |
| Actin-F | SEQ ID NO: 19 | GCTGATAGGATGAGCAAGGAG |
| Actin-R | SEQ ID NO: 20 | GAGCCTCCAATCCAGCAGACACTAT |

The relative expression level of MfERF026 was calculated based on the RT-qPCR results using a 2^{-ΔΔCt} method.

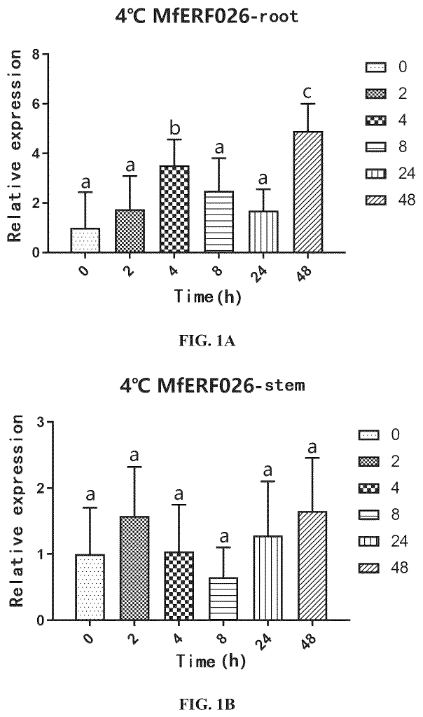

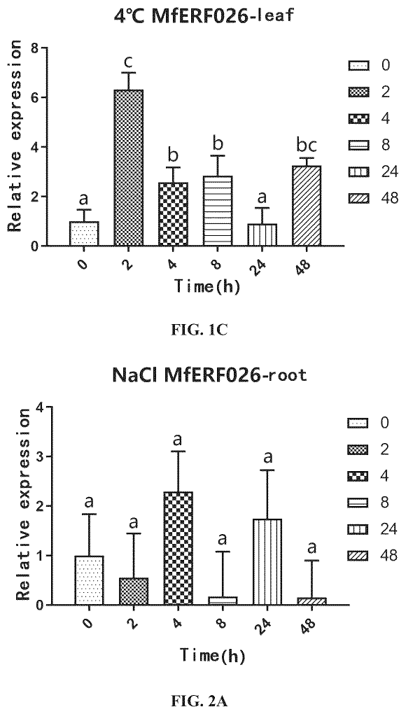

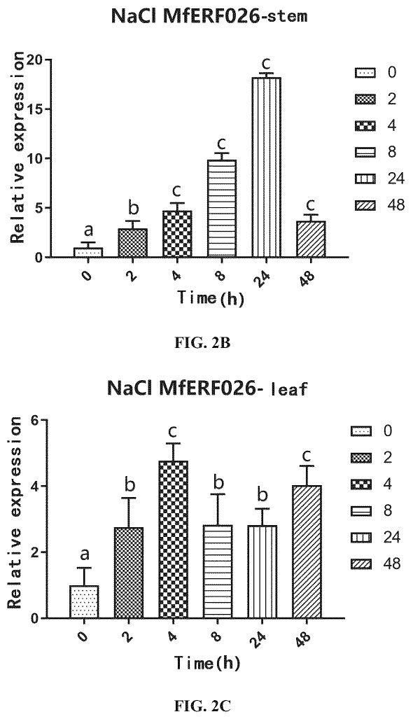

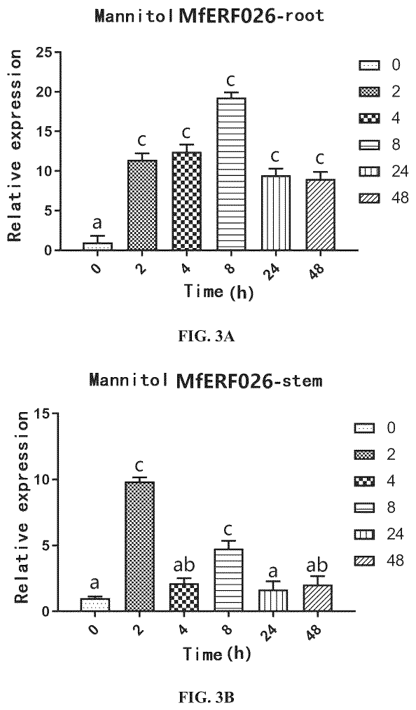

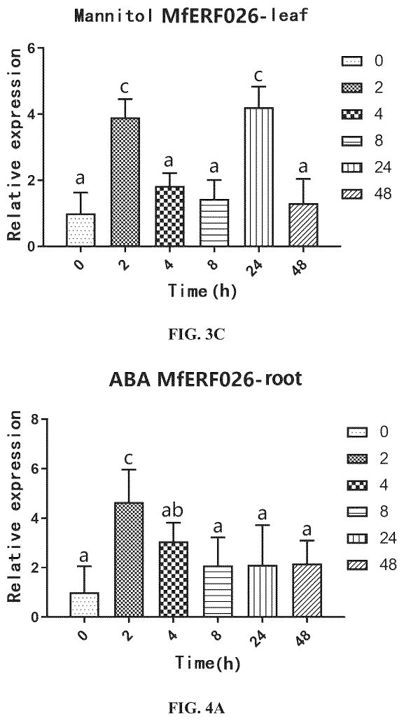

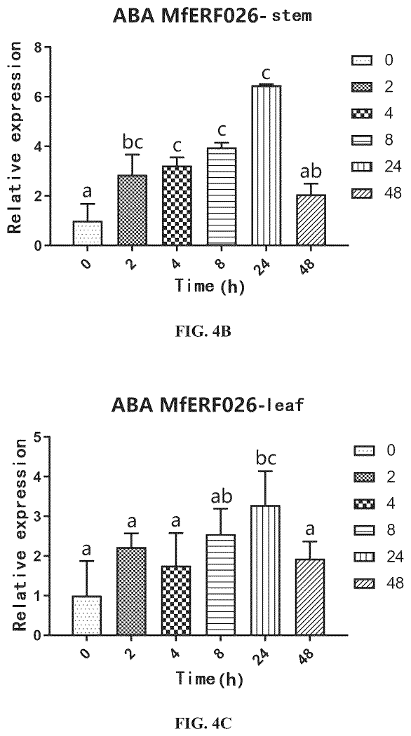

FIGS. 1A-IC show the relative expression of MfERF026 under cold stress conditions obtained by RT-qPCR. FIGS. 2A-2C shows the relative expression of MfERF026 under salt stress conditions obtained by RT-qPCR. FIGS. 3A-3C shows the relative expression of MfERF026 under drought stress conditions obtained by RT-qPCR. FIGS. 4A-4C shows the relative expression of MfERF026 under ABA stress conditions obtained by RT-qPCR.

As shown in FIG. 1A to FIG. 4C, MfERF026 was highly expressed under low temperature, salt, drought, and ABA treatments. The expression level reached maximum under drought stress, indicating a significant response to osmotic stress. The expression levels in plant roots, stems, and leaves were different, and their expression levels increased or decreased significantly with time. These results proved that MfERF026 responded to the low temperature, salt, drought, and ABA treatments.

### Example 3

Analysis of Subcellular Localization of MfERF026 in M. falcata
1. Construction of Recombinant Plasmid pBE-GFP-MfERF026

The M. falcata cDNA was obtained using the method in Example 1.

The MfERF026 cDNA was ligated into a pEASY®-Blunt vector according to the instructions of a pEASY®-Blunt kit (CB101-01) of TransGen Biotech.

PCR was conducted using a primer pair of MfERF026 PBE-F and MfERF026 PBE-R to obtain the full-length cDNA of MfERF026 with BamHI and SalI restriction sites. The primer sequences for the primer pair of MfERF026 PBE-F and MfERF026 PBE-R are shown in Table 3.

TABLE 3
Primer information for MfERF026 PBE-F and MfERF026 PBE-R

| Primer name | Primer ID | 5′-3′ |
|---|---|---|
| MfERF026 | SEQ ID | GACTCTAGAGCAGTCGACGATGAATAGTAGTACTATTGAAC |
| PBE-F | NO: 7 | |
| MfERF026 | SEQ ID | GGCGACCGGTGGATCCCGGCTATAGTTATTATAAATGTCAC |
| PBE-R | NO: 8 | |

The PCR system is shown in Table 4.

TABLE 4
PCR system

| Reagent | Consumption (μL) |
|---|---|
| 5xPrime STAR GXL Buffer | 10.0 |
| dNTP Mixture (2.5 mmol/L) | 4.0 |
| MfERF026 PBE-F(5 μmoL) | 2.0 |
| MfERF026 PBE-R(5 μmoL) | 2.0 |
| Prime STAR GXL DNA Ploymerase | 1.0 |
| pEASY ®-Blunt-MfERF026 (100 ng/μL) | 1.0 |
| ddH₂O | Supplementing to 50.0 μL |

PCR conditions included: initial denaturation at 98° C. for 5 min; denaturation at 98° C. for 10 s; annealing at 60° C. for 30; extension at 68° C. for 1 mo, 30 cycles; post-extension at 68° C. for 10 min; and storage at 4 (C.

After the PCR was completed, agarose gel electrophoresis was conducted to detect the PCR products, and a target fragment was obtained by determining the size of the band.

After the PCR was completed, a target fragment (about 470 bp) of MERF026 was recovered using a SanPrep column DNA gel recovery kit of Sangon Biotech Co., Ltd. according to the instructions of the kit.

A pBE empty plasmid was double-digested with BamHI and SalI using an enzyme digestion system shown in Table 5.

TABLE 5
Enzyme digestion system

| Reagent | Consumption (μL) |
|---|---|
| pBE plasmid (100 ng/μL) | 10 |
| 10xT Buffer | 3 |
| BcmH I | 1 |
| Sal I | 1 |
| ddH₂O | Supplementing to 20 μL |

The enzyme digestion system was mixed for digestion at 37° C. for 1 h. After the enzyme digestion, a resulting linearized plasmid vector was recovered using the SanPrep column DNA gel recovery kit of Sangon Biotech Co., Ltd. according to the instructions of the kit.

The homologous recombination ligation of the target fragment and the pBE vector was conducted using TaKaRa In-Fusion® HD Cloning Kit. An obtained ligation product was transformed into E. coli DH5a, tested by colony PCR, and sent for sequencing. The obtained positive clones verified by sequencing were subjected to expanded culture, and plasmids were extracted for later use.

2. Transformation of Agrobacterium GV3101 with Recombinant Plasmid pBE-GFP-MfERF026

5 μL of the recombinant plasmid pBE-GFP-MfERF026 at a concentration of 100 ng/μL and empty pBE-GFP were added to 50 μL of Agrobacterium GV3101 competent cells. After gently mixing, the cells were placed on ice for 5 min, in liquid nitrogen for 5 min, transferred to a 37° C. water bath for heat shock for 5 min, and then in ice water bath for 5 min. The cells were added to 700 μL of LB liquid medium balanced to room temperature on a clean workbench, and cultured at 28° C. and 200 rpm for 2 h.

After the culture was completed, the cells were centrifuged at 6,000 rpm for 1 min, 500 μL of the supernatant was discarded, and the remaining liquid was pipetted to resuspend the cells and facilitate spreading in the next step. 80 μL of the remaining liquid was spread on LB solid medium containing 50 mg/L Kan (kanamycin), 50 mg/L genta (gentamicin), and 25 mg/L Rif (rifampicin antibiotic), and then incubated upside down at 28° C. for 36 h to 48 h until a single colony grew.

3. Injection into the Lower Epidermis of Nicotiana Benthamiana Leaves

A single colony of positive Agrobacterium GV3101 was picked in YEB liquid medium containing Kan, Gent, and Rif, and cultured with shaking at 28° C. for 24 h.

After the culture was completed, 500 μL of the bacterial solution was added into 40 mL of triple antibody (Kan, Gent, and Rif)-containing YEB liquid medium containing 500 μL of 1 M 2-(N-morphine)ethanesulfonic acid (MES) and 5 μL of 200 mM acetosyringone (AS, which facilitated Agrobacterium infection of tobacco leaves), and cultured with shaking at 28° C. until the OD₆₀₀ value was at 1.2 to 1.5.

After the culture was completed, the cells were collected by centrifugation (4° C., 8,000 rpm, 10 min), added with an infection solution (where each 100 mL of ddH₂O included 1 mL of 1 M MES, 1 mL of 1 M MgCl₂, and 100 μL of 200 mM AS), resuspended until the OD₆₀₀ value was 1.0, allowed to stand in the dark for 4 h, then injected into the lower epidermis of 4-week-old tobacco leaves, and cultured in a plant tissue culture lab for 36 h.

4. Observation of Leaf Fluorescence with Laser Confocal Microscope

The tobacco leaves were cut at the infected site and placed in a petri dish, DAPI was added dropwise on the leaves to allow staining for 20 min, and washed twice with PBS for 20 min each time. The stained leaves were placed face up on a glass slide and observed with a laser confocal microscope.

FIG. 5 shows the observation results under a laser confocal microscope in the subcellular localization experiment.

As shown in FIG. 5, the fluorescence signal of GFP fused to MfERF026 co-localized with the DAPI, indicating that MfERF026 was located in the nucleus.

### Example 4

Analysis of Transcriptional Activation Experiment of MfERF026 n M. falcata

The M. falcata cDNA was obtained using the method in Example 1.

The cDNA of MfERF026 was ligated into a pEASY®-Blunt vector according to the instructions of a pEASY®-Blunt kit (CB101-01) of TransGen Biotech.

5 fragments of MfERF026 were obtained using different primers, and included: full length (a), N-terminal+ERF domain (b), ERF domain (c), ERF domain+C-terminal (d), and C-terminal (e), and primer information is shown in Table 6.

TABLE 6
Primer encoding 5 fragments of MfERF026: full length, N-terminal + ERF domain, ERF
domain, ERF domain + C-terminal, and C-terminal

| Target fragment | Primer name | Primer ID | 5′-3′ |
|---|---|---|---|
| MfERF026-a | MfERF026a-F | SEQ ID NO: 9 | CGGGATCCATGAATAGTAGTACTATTGAAC |
| | MfERF026a-R | SEQ ID NO: 10 | ATAAGAATGCGGCCGCCTAGCTATAGTTATTATAAATG |
| MfERF026-b | MfERF026a-F | SEQ ID NO: 9 | CGGGATCCATGAATAGTAGTACTATTGAAC |
| | MfERF026b-R | SEQ ID NO: 11 | ATAAGAATGCGGCCGCAAGCAAGTTTACTAACTCAG |
| MfERF026-c | MfERF026c-F | SEQ ID NO: 12 | CGGGATCCGTATACCGTGGTGTCCG |
| | MfERF026b-R | SEQ ID NO: 11 | ATAAGAATGCGGCCGCAAGCAAGTTTACTAACTCAG |
| MfERF026-d | MfERF026c-F | SEQ ID NO: 12 | CGGGATCCGTATACCGTGGTGTCCG |
| | MfERF026a-R | SEQ ID NO: 10 | ATAAGAATGCGGCCGCCTAGCTATAGTTATTATAAATG |
| MfERF026-e | MfERF026e-F | SEQ ID NO: 13 | CGGGATCCCCTCGTCCGGCTTCACTC |
| | MfERF026a-R | SEQ ID NO: 10 | ATAAGAATGCGGCCGCCTAGCTATAGTTATTATAAATG |

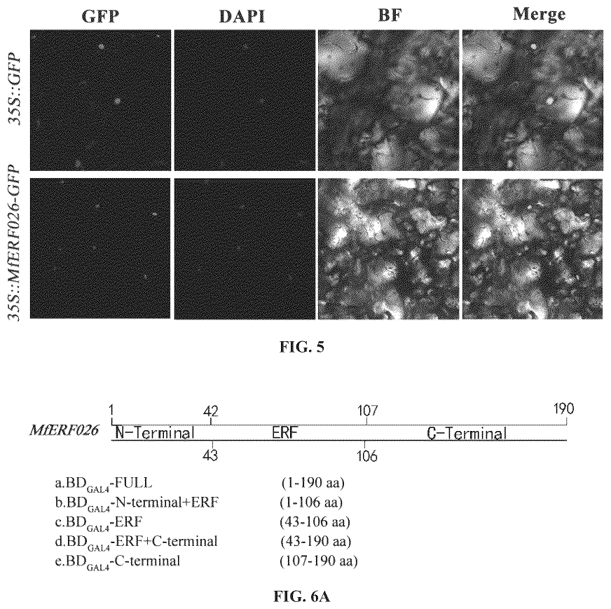

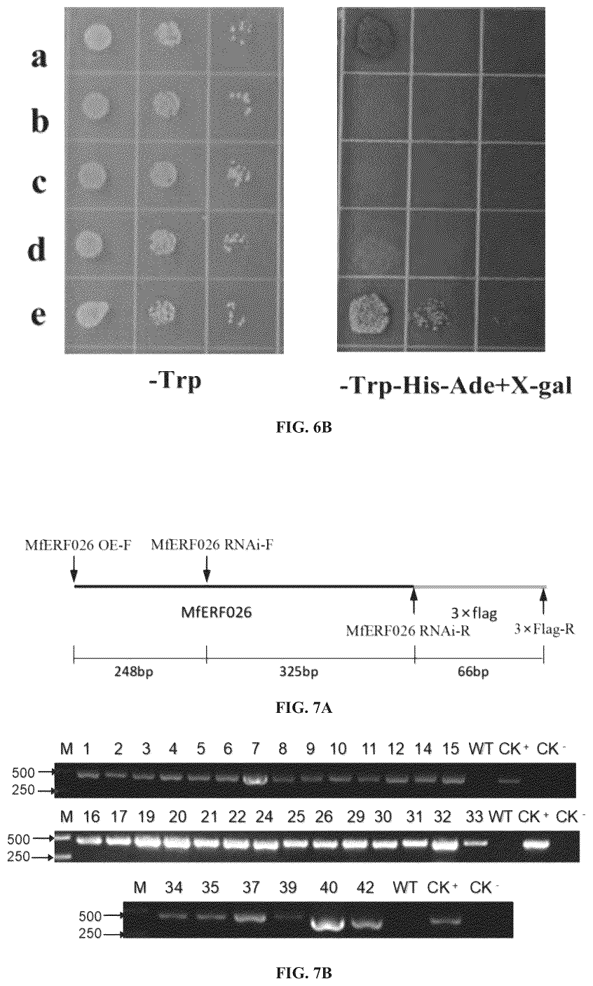

PCR was conducted using the primers shown in Table 6 separately to obtain the 5 fragments of MfERF026: full length (a), N-terminal+ERF domain (b), ERF domain (c), ERF domain+C-terminal (d), and C-terminal (e). The details of the 5 fragments are shown in FIG. 6A.

The PCR system is shown in Table 7.

TABLE 7
PCR system

| Reagent | Consumption (μL) |
|---|---|
| pEASY ®-Blunt-MfERF026 | 2 |
| plasmid (100 ng/μL) | |
| 5xPrime STAR GXL Buffer | 10 |
| Prime STAR GXL DNA Ploymerase | 1 |
| dNTP Mixture(2.5 mmol/L) | 4 |
| PF(5 μmol/L) | 3 |
| PF(5 μmol/L) | 3 |
| RNase free ddH₂O | Supplementing to 50.0 μL |

PCR conditions included: initial denaturation at 98° C. for 5 min; denaturation at 98° C. for 30 s; annealing at 60° C. for 30 s; extension at 68° C. for 1 min and 30 s, 30 cycles; post-extension at 68° C. for 10 min; and storage at 16° C. for 10 min.

The 5 fragments of MfERF026: full length (a), N-terminal+ERF domain (b), ERF domain (c), ERF domain+C-terminal (d), and C-terminal (e) were inserted into a pGBKT7 vector separately to construct a fusion expression vector.

### Construction of Fusion Expression Vector

(1) After the PCR was completed, a target fragment was recovered using a SanPrep column DNA gel recovery kit of Sangon Biotech Co., Ltd., which was performed according to the instructions of the kit.

(2) A pBE empty plasmid was double-digested with BamHI and Nod using an enzyme digestion system shown in Table 8.

TABLE 8
Enzyme digestion system

| Reagent | Consumption (μL) |
|---|---|
| pGBKT7 plasmid (100 ng/μL) | 10 |
| 10xKbuffer | 1 |
| BamHI(15 U/μL) | 1 |
| NotI(10 U/μL) | 1 |
| ddH₂O | Supplementing to 20 μL |

The enzyme digestion system was mixed for digestion at 37° C. for 1 h. After the PCR was completed, a linearized plasmid vector was recovered using a SanPrep column DNA gel recovery kit of Sangon Biotech Co., Ltd., which was performed according to the instructions of the kit.

(3) According to a molar ratio of the target fragment to the vector at (3-10):1, the target gene fragment was ligated to the pGBKT7 linearized vector, transformed into E. coli competent cells DH5α, spread onto a plate, and positive clones were selected to allow PCR detection. The obtained positive clones verified by sequencing were subjected to expanded culture, and a plasmid was extracted for later use.

5 obtained fusion expression vectors were transferred into AH109 yeast separately, and yeast transformants of the 5 fusion expression vectors were separately cultured in an SD/-Trp medium (i.e., yeast-deficient medium, SD medium lacked tryptophan) and an X-α-gal-added SD/-Trp/-His/-Ade medium (i.e., the SD medium lacked tryptophan, histidine, and adenine).

The culture results of the yeast transformants of the 5 fusion expression vectors on different media are shown in FIG. 6B.

As shown in FIG. 6, the 5 yeast transformants of fusion expression vectors grew well on SD/-Trp medium. On the SD/-Trp/-His/-Ade medium supplemented with X-α-gal, only blue yeast colonies were observed in a and e. This indicated that the β-galactosidase reporter gene had been transcriptionally activated and exhibited a desirable activity. The results proved that the MfERF026 protein had a transcription activation activity, and its transcription activation domain was located at the C-terminal.

### Example 5

Obtaining Transgenic M. sativa with Inhibited Expression of MfERF026
1. Construction of Inhibition Expression Vector pCAM1307-RNAi-MfERF026

The M. falcata cDNA was obtained using the method in Example 1.

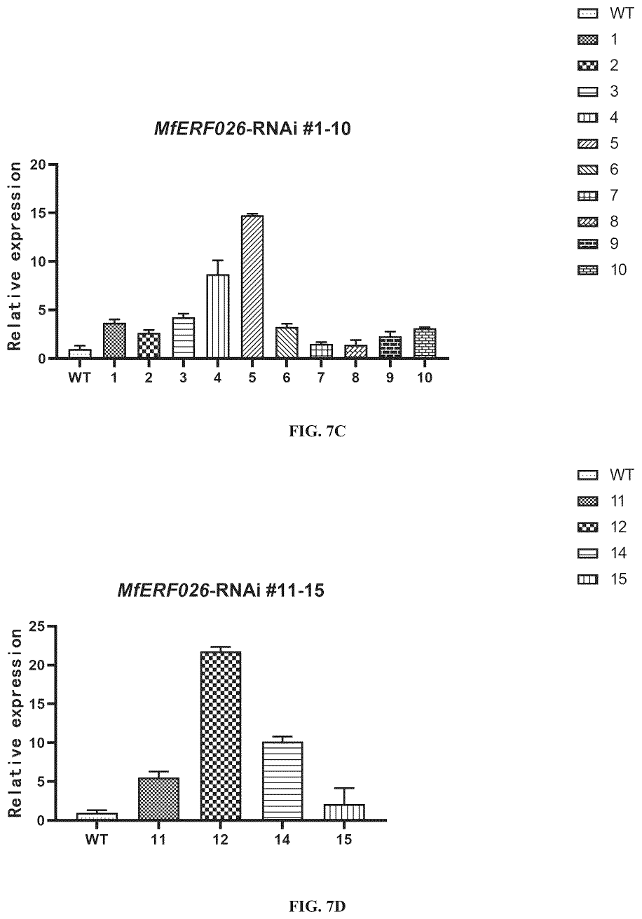

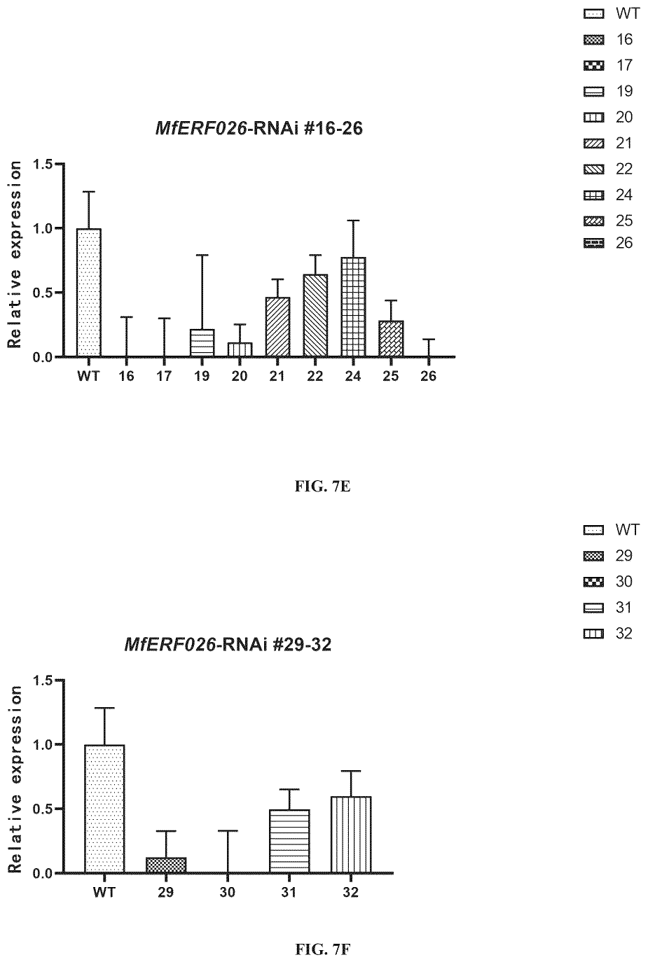

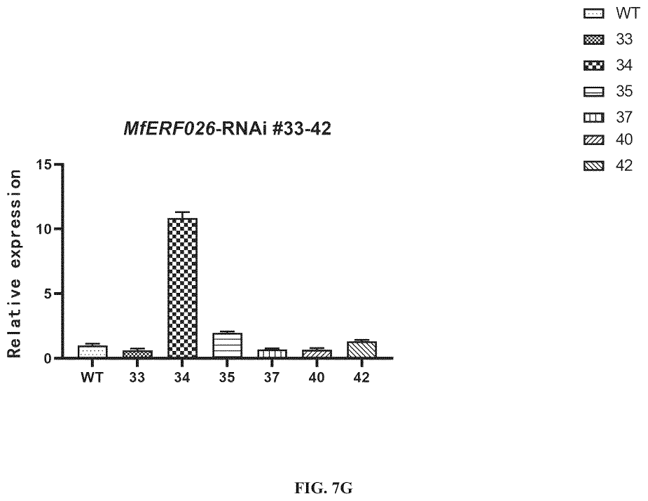

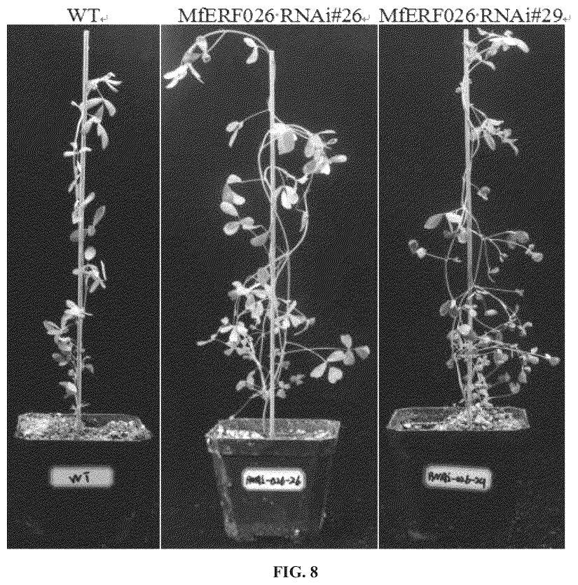

PCR was conducted using MfERF026 RNAi-F and MfERF026 RNAi-R (the primer amplification fragments are shown in FIG. 7A), to obtain a full-length cDNA of RNAi-MfERF026 with BamHI and XbaI restriction sites. The PCR system and PCR conditions were the same as those in Example 3. The information of primer pair including MfERF026 RNAi-F and MfERF026 RNAi-R is shown in Table 9, and the amplified target fragment is set forth in SEQ ID NO: 17.

TABLE 9
Sequence information of primers MfERF026 RNAi-F and MfERF026 RNAi-R

| Primer name | Primer ID | 5′-3′ |
|---|---|---|
| MfERF026 RNAi-F | SEQ ID NO: 14 | CGGGATCCTTCAGAGGTGAAGACACACGTAGATGTAGCTGCT |
| | | CTTAGTAT |
| MfERF026 RNAi-R | SEQ ID NO: 15 | GCTCTAGACTAGCTATAGTTATTATAAATG |

After the PCR was completed, the agarose gel electrophoresis was conducted to detect the PCR products, and a target fragment was obtained by determining the size of the band.

After the PCR was completed, a target fragment (about 330 bp) was recovered using a SanPrep column DNA gel recovery kit of Sangon Biotech Co., Ltd., and the operations were done according to the instructions of the kit.

An empty plasmid pCAM1307 (hereinafter referred to as p1307) was digested with BamHI and XbaI, and the enzyme digestion system is shown in Table 10.

TABLE 10
Enzyme digestion system

| Reagent | Consumption (μL) |
|---|---|
| p1307 plasmid (100 ng/μL) | 10 |
| 10xK Buffer | 1 |
| BamH I | 1 |
| XbaI | 1 |
| ddH₂O | Supplementing to 20 μL |

The enzyme digestion system was mixed for digestion at 37° C. for 1 h. After the PCR was completed, a linearized plasmid vector was recovered using a SanPrep column DNA gel recovery kit of Sangon Biotech Co., Ltd., and the operations were done according to the instructions of the kit.

According to a molar ratio of the target fragment to the vector at (3-10):1, the target gene fragment was ligated to the p1307 linearized vector, transformed into E. coli competent cells DH5α, spread onto a plate, and positive clones were selected to allow PCR detection. The obtained positive clones verified by sequencing were subjected to expanded culture, and a plasmid was extracted for later use.

2. Transformation of Agrobacterium EHA105 with the Inhibition Expression Vector pCAM1307-RNAi-MfERF026

5 μL of the inhibition expression vector pCAM1307-RNAi-MfERF026 at a concentration of 100 ng/μL was pipetted and added to 50 μL of EHA105 competent cells. After snap-freezen in liquid nitrogen for 2 min and heat shocked at 42° C. for 30 s, the cells were added into 600 μL of a YEB liquid medium and cultured at 28° C. and 190 rpm for 4 h.

After the culture was completed, 80 μL of a resulting bacterial solution was spread on a YEB solid medium containing kanamycin (50 mg/L) and rifampicin (75 mg/L), and cultured upside down at 28° C. for 2 d.

After the culture was completed, positive colonies are selected. The selected positive single colonies were subjected to expanded culture until the OD₆₀₀ value was 0.6 to 0.8, an obtained Agrobacterium bacteria solution was centrifuged at 2,400 rpm at room temperature for 15 min, a supernatant was discarded, and the cells were suspended until the OD₆₀₀ value reached 0.2 to 0.3 to prepare an infection solution for later use.

3. Agrobacterium EHA105-Mediated Leaf Disk Transformation of M. sativa

The M. sativa “Zhongmu No. 1” was selected as a plant material, and the M. sativa leaves in desirable growth condition for four to six weeks were selected as an infection material.

The M. sativa was transformed by an EHA105-mediated leaf disc method:

After the infection material was washed with ultrapure water, an appropriate amount of a mixed solution including hypochloric acid rinsing solution and Tween-20 was added (the Tween-20 and hypochloric acid rinsing solution in the mixed solution were at a volume ratio of 1:3000; the hypochloric acid in the hypochloric acid rinsing solution had a mass percentage of 7%; and the hypochloric acid rinsing solution was purchased from Taobao (sterilization 99.99%, imported Clorox, 1 L*2 bottle, household indoor clothing disinfectant, hypochloric acid disinfection bleach), such that the mixed solution covered the leaves to allow sterilization for 10 min to 13 min.

The infection solution prepared in step 2 was mixed with the sterilized trifoliate plant, the leaves were infiltrated at 0.08 MPa to 0.09 MPa under vacuum for 10 min, ultrasonicated (40 kHz) at room temperature for 4 min to 5 min, and infiltrated again at 0.08 MPa to 0.09 MPa under vacuum 10 min.

After the vacuum infiltration was completed, the Agrobacterium suspension was discarded, the Agrobacterium on the leaf surface was removed by absorbing with sterilized filter paper, the treated trifoliate plant was spread on the co-culture medium, and cultured in the dark for 24 h to 30 h. The obtained explants were transplanted into a selective medium to allow callus induction culture, where subculture was conducted every 2 weeks for 5 to 6 weeks.

The calli in desirable growth conditions were transferred to an MSBK medium and cultured for 10 d to 14 d until green embryoid appeared.

The calli with green embryoid were transferred into an SH9 medium to allow shoot differentiation. Subculture was conducted every 3 to 4 weeks until the shoots grew up, which generally took 6 to 8 weeks on this medium. When the shoots grew 2 to 3 fully expanded trifoliate leaves, the shoots were transferred into an MSO medium for rooting culture to obtain rooted plants (if the strong-growing seedlings were difficult to take root within a month, 1 mg/L IAA was added).

The rooted plants were planted into a matrix including vermiculite and perlite at a volume ratio of 1:1 to allow acclimatization. Surviving plants were transferred to a medium with nutrient soil and vermiculite at a volume ratio of 1:1 for culture.

4. Detection of transgenic M. sativa

The transgenic lines were verified using PCR and Q-PCR.

In this step, wild-type M. sativa plants were used as controls, where control experimental material was the leaf tissues of wild-type M. sativa “Zhongmu No. 1” grown normally for 30 d, and their treatment and detection methods were the same as those of the transgenic plants.

DNA from leaves of transgenic plants and leaves of wild plants that had survived for 30 d were obtained using the CTAB method to allow PCR identification.

The PCR was conducted using MfERF026 RNAi-F and 3×Flag-R primers (the primer amplification fragment is shown in FIG. 7A). The information of primers MfERF026 RNAi-F and 3×Flag-R is shown in Table 11.

TABLE 11
Primer information

| Primer name | Primer ID | 5′-3′ |
|---|---|---|
| MfERF026 RNAi-F | SEQ ID NO: 14 | CGGGATCCTTCAGAGGTGAAGACACACGTAGATGTAGCTGCTC |
| | | TTAGTAT |
| 3xFlag-R | SEQ ID NO: 16 | CTACTTATCGTCATCGTCCTTGTA |

The PCR system is shown in Table 12.

TABLE 12
PCR system

| Reagent name | Consumption (μL) |
|---|---|
| 10x Buffer | 2.5 |
| dNTPs (2.5 mM each) | 2.0 |
| MfERF026 RNAi-F | 3.0 |
| 3xflag-PR | 3.0 |
| DNA | 2 |
| rTaqDNA Polymerase(5 U/μL) | 0.125 |
| ddH₂O | Supplementing to 25 μL |

PCR conditions included: initial denaturation at 98° C. for 5 min; denaturation at 98° C. for 10 s; annealing at 57° C. for 30 s; extension at 68° C. for 1 min, 30 cycles; post-extension at 68° C. for 10 min; and storage at 4° C.

After the PCR was completed and the PCR product was obtained, agarose gel electrophoresis was conducted to detect whether a target band was produced and a size of the target band, so as to determine the positive strain.

The gel electrophoresis pattern of the PCR product is shown in FIG. 7B.

RNA was extracted from the leaves of the lines that were initially detected as positive and reverse transcribed into cDNA, and then fluorescence quantitative RT-qPCR was conducted to calculate the relative expression level of MfERF026 in each line. Wild-type M. sativa RNA at the same growth period was extracted and reverse transcribed into cDNA, and quantitative RT-qPCR was conducted to calculate the relative expression level of MfERF026 as a control group.

RT-qPCR was conducted using the MfERF026 DL-F and MfERF026 DL-R as primers according to the instructions of a Reverse Transcription Kit (#RR047Q, TAKARA Biotechnology). The information of primers MfERF026 DL-F and MfERF026 DL-R is shown in Table 2.

The relative expression level of MfERF026 was calculated based on the RT-qPCR results, and the calculation method and reference gene were the same as those in Example 2.

The RT-qPCR detection results of MfERF026 in transgenic M. sativa and wild-type M. sativa are shown in Table 13 and FIGS. 7C-7G.

TABLE 13
RT-qPCR detection results of MfERF026 in transgenic M. sativa and wild-type M. sativa

| Root system ID | WT | 1 | 2 | 3 | 4 | 5 | |
|---|---|---|---|---|---|---|---|
| Relative | 1 | 3.696395702 | 2.666397516 | 4.272497773 | 8.705489094 | 14.75885607 | |
| expression level | | | | | | | |
| Root system ID | WT | 6 | 7 | 8 | 9 | 10 | |
| Relative | 1 | 3.272519645 | 1.548315837 | 1.435649964 | 2.310759365 | 3.128273539 | |
| expression level | | | | | | | |
| Root system ID | WT | 11 | 12 | 14 | 15 | | |
| Relative | 1 | 5.539331733 | 21.79154398 | 10.15253741 | 2.137380309 | | |
| expression level | | | | | | | |
| Root system ID | WT | 16 | 17 | 19 | 20 | 21 | |
| Relative | 1 | 0.000170468 | 0.000722038 | 0.219165015 | 0.114911495 | 0.466560473 | |
| expression level | | | | | | | |
| Root system ID | WT | 22 | 24 | 25 | 26 | | |
| Relative | 1 | 0.645638668 | 0.776569093 | 0.284394388 | 0.000189571 | | |
| expression level | | | | | | | |
| Plant line ID | WT | 29 | 30 | 31 | 32 | | |
| Relative | 1 | 0.125675276 | 0.00035771 | 0.498250989 | 0.600333031 | | |
| expression level | | | | | | | |
| Root system ID | WT | 33 | 34 | 35 | 37 | 40 | 42 |
| Relative | 1 | 0.635141522 | 10.83997828 | 1.969879598 | 0.700018392 | 0.662697461 | 1.319191848 |
| expression level | | | | | | | |

Note:
WT refers to wild-type

According to the quantitative results shown in Table 13 and FIG. 7C-7G, MfERF026 expression of the transgenic lines was significantly reduced compared with that of the wild-type (WT). The results proved that MfERF026 was transferred into the M. sativa genome and showed inhibited expression. Transgenic lines #16, #17, #26, and #29 showed the best inhibitory effects, so the lines #16, #17, #26, and #29 were selected for subsequent experiments.

6. Developmental Phenotypes, Drought Tolerance Experiments, and Cold Tolerance Experiments of Transgenic Lines with Inhibited Expression
(1) Cuttings of Transgenic M. sativa

On the basis of Experiment 5, the stems and leaves of transgenic lines #16, #17, #26, and #29 with desirable growth status were selected, their branches about 7 cm long were cut off at an angle, and a bottom incision of each branch was dipped into a rooting powder (Solarbio, Cat. No. R8240) solution (1 g/L) for 10 min, inserted into the thoroughly poured vermiculite, leaving only the nodes on the surface. White and strong roots grew in about 2 weeks, and were transplanted into a matrix with vermiculite and nutrient soil at a ratio of 3:2 (v/v), while the wild-type M. sativa from the same batch were also cut as a control group. The number of test samples in each group was >30.

(2) Developmental Phenotypes of Transgenic Lines with Inhibited Expression

Wild-type plants and transgenic plants grown for 30 d in a medium with vermiculite and nutrient soil at a ratio of 3:2 (v/v) were selected for measurement, statistics, and analysis of the developmental phenotypes.

FIG. 8 showed the growth of transgenic lines #26 and #29 and wild-type M. sativa.

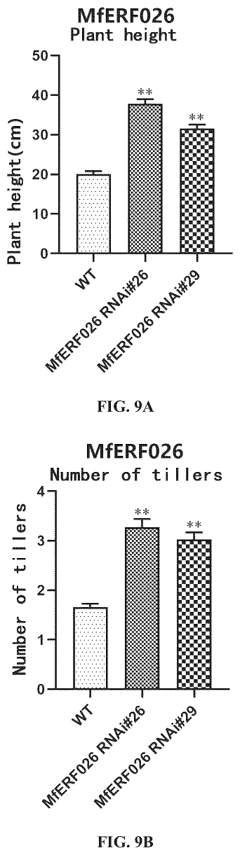

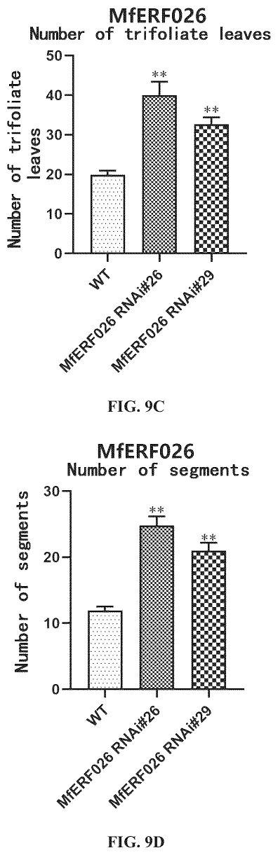

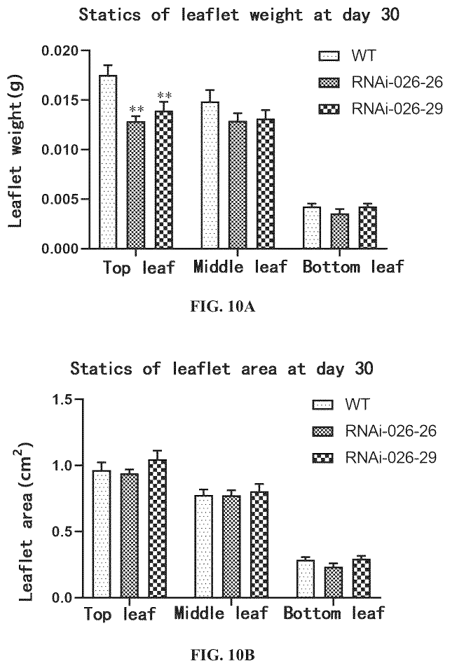

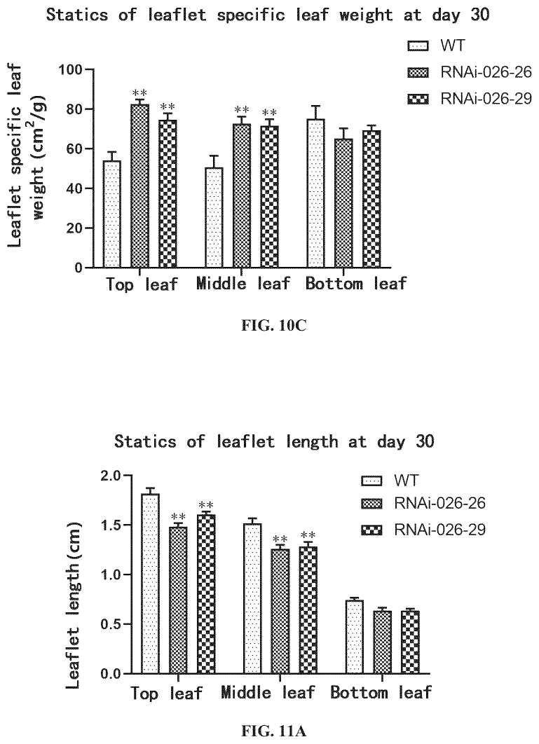

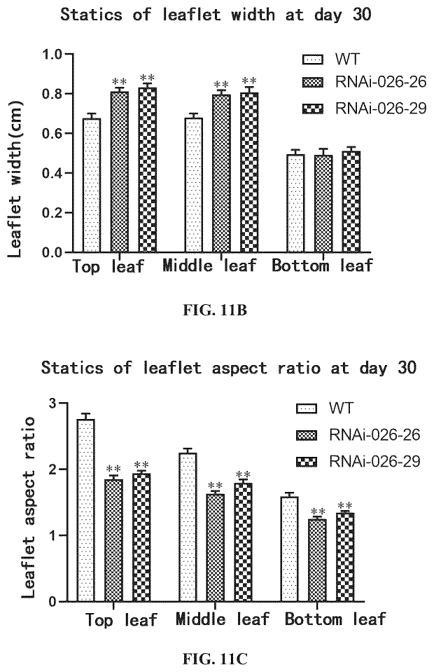

The statistical results of developmental phenotypes of wild-type plants and transgenic plants are shown in Tables 14 to 15 and FIG. 9 to FIG. 11C. The plant height, number of tillers, number of trifoliate leaves, and number of segments of wild-type plants and transgenic plants are shown in Table 14 and FIGS. 9A-9D. The statistical results of leaflet weight, leaflet area, and leaflet specific leaf weight (i.e., the ratio of leaflet weight to leaflet area) of wild-type plants and transgenic plants are shown in Table 15 and FIGS. A-C. The leaflet length, leaflet width, and leaflet aspect ratio of wild-type plants and transgenic plants are shown in Table 15 and FIGS. 11A-11C.

TABLE 14
Plant height, number of tillers, number of trifoliate leaves, and
number of segments of wild-type plants and transgenic plants

| | Number of | Number of | Plant | Number of |
|---|---|---|---|---|
| Group | segments | tillers | height | trifoliate leaves |
| WI | 11.96 | 1.663 | 20.05 | 19.94 |
| MfERF026 RNAi#26 | 24.79 | 3.274 | 37.84 | 40.04 |
| MfERF026 RNAi#29 | 21.02 | 3.025 | 31.59 | 32.63 |

TABLE 15
Statistical results of leaflet of wild-type plants and transgenic plants

| | | | | Specific | | | Leaf |
|---|---|---|---|---|---|---|---|
| | | Leaf | Leaf | leaf | Leaf | Leaf | aspect |
| Group | Leaf type | weight | area | weight | length | width | ratio |
| WT | Top leaf | 0.01754 | 0.9659 | 54.16 | 1.818 | 0.6783 | 2.762 |
| | Middle leaf | 0.01489 | 0.7766 | 50.7 | 1.518 | 0.6806 | 2.251 |
| | Bottom leaf | 0.004273 | 0.288 | 75.18 | 0.7441 | 0.4953 | 1.59 |
| MfERF026 RNAi#26 | Top leaf | 0.01286 | 0.9396 | 82.58 | 1.486 | 0.8125 | 1.851 |
| | Middle leaf | 0.01291 | 0.7751 | 72.83 | 1.261 | 0.7974 | 1.631 |
| | Bottom leaf | 0.00356 | 0.2338 | 65.17 | 0.6358 | 0.4916 | 1.25 |
| MfERF026 RNAi#29 | Top leaf | 0.01395 | 1.049 | 74.77 | 1.607 | 0.8319 | 1.942 |
| | Middle leaf | 0.01312 | 0.806 | 71.72 | 1.285 | 0.8073 | 1.795 |
| | Bottom leaf | 0.004273 | 0.2943 | 69.45 | 0.6362 | 0.5111 | 1.344 |

As shown in Tables 14 and 15 and FIG. 8 to FIG. 11C, compared with the control (WT) plants, the plants with inhibited expression had increased plant height, increased number of tillers, and increased number of segments and number of trifoliate leaves. Compared with the control (WT) plants, the plants with inhibited expression had reduced top leaf weight, reduced leaflet length but increased leaflet width, no significant difference in leaf area, increased leaflet specific leaf weight, and decreased aspect ratio.

(3) Drought Tolerance Experiment of Transgenic Lines with Inhibited Expression

The wild-type plants and transgenic lines #16, #17, #26, and #29 that were grown for 30 d in a soil matrix with vermiculite and nutrient soil a volume ratio of 3:2 were saturated and the drought tolerance experiment was started, where watering was stopped for 3 weeks and resumed for 1 week. After the rewatering was completed, the survival rate of the M. sativa in the experimental group and the control group was calculated, and the changes in electrical conductivity and physiological and biochemical indicators of the M. sativa in the experimental group and the control group before and after the experiment were detected and compared. #16 had 20 test plants; #17, #26, and #29 had 21 test plants separately; wild-type (WT) had 26 test plants.

Method for measuring conductivity: i. the leaves to be measured were placed in a 50 mL centrifuge tube containing 25 mL of ultrapure water; ii. the centrifuge tube was placed into a vacuum pump, vacuumized (<0.09 MPa) for 15 min, and then transferred into a shaker, and balanced by shaking at 230 rpm at room temperature for 1 h; iii. the conductivity meter was used to measure the electric conductivity of the liquid in the centrifuge tube, and an initial value S1 was recorded; iv. the centrifuge tube containing the sample was treated in a boiling water bath tank for 15 min, and then transferred into a shaker, balanced by shaking at 230 rpm at room temperature for 1 h, and the liquid in the tube was cooled naturally to room temperature; v. the electric conductivity of the liquid in the centrifuge tube was measured and the value was recorded as S2; vi. the electric conductivity of ultrapure water was measured at the same time, and the value was recorded as S0; vii. a relative electric conductivity of the sample leaves was measured based on S1 and S2 of each sample and the electric conductivity S0 of ultrapure water. The calculation formula of electric conductivity was: relative electric conductivity=(S1-S0)/(S2-S0).

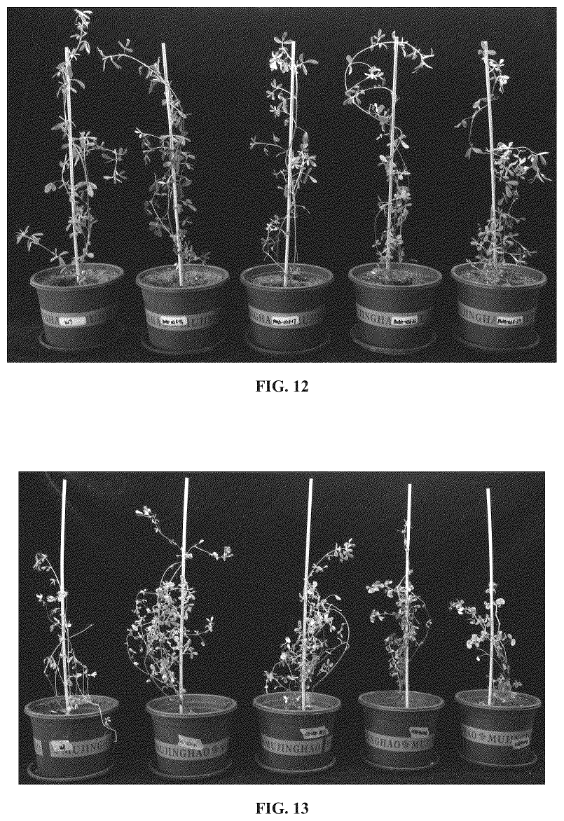

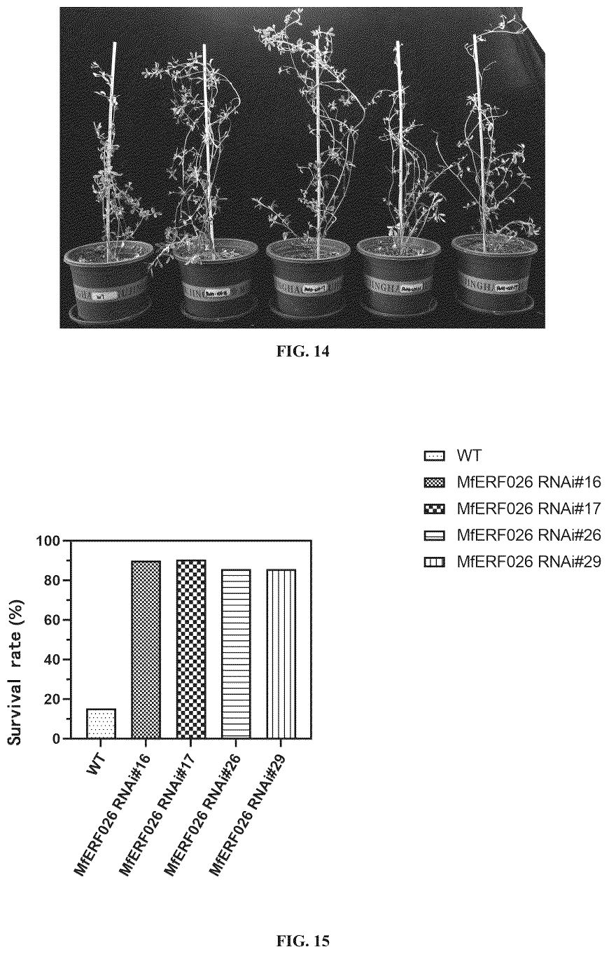

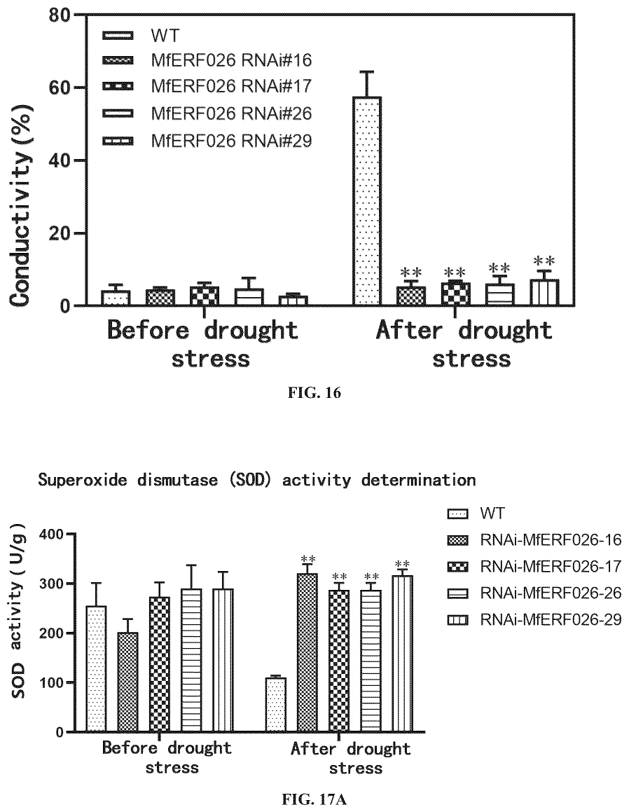

The drought tolerance test results of wild-type plants and transgenic lines are shown in Tables 16 and 17 and FIG. 12 to FIG. 16. FIG. 12 showed the growth status of wild-type plants and transgenic plants before the drought tolerance test; FIG. 13 shows the growth status of wild-type plants and transgenic plants under drought stress for 3 weeks; FIG. 14 shows the growth status of wild-type plants and transgenic plants after one week of rewatering. FIG. 12 to FIG. 14 show the wild-type plants, transgenic plant #16, transgenic plant #17, transgenic plant #26, and transgenic plant #29 from left to right in sequence. The statistical results of survival rate after drought stress are shown in Table 16 and FIG. 15; the statistical results of electrical conductivity before and after drought stress are shown in Table 17 and FIG. 16.

TABLE 16
Statistical results of drought tolerance survival
rates of wild-type plants and transgenic lines

| Group | Survival rate |
|---|---|
| WT | 15.38% |
| MfERF026 RNAi#16 | 90% |
| MfERF026 RNAi#17 | 90.48% |
| MfERF026 RNAi#26 | 85.71% |
| MfERF026 RNAi#29 | 85.71% |

TABLE 17
Statistical results of drought tolerance electric conductivity
of wild-type plants and transgenic lines

Electric conductivity

| Group | Before stress | After stress |
|---|---|---|
| WT | 4.35 | 57.68 |
| MfERF026 RNAi#16 | 4.618 | 5.402 |
| MfERF026 RNAi#17 | 5.4 | 6.42 |
| MfERF026 RNAi#26 | 4.928 | 6.204 |
| MfERF026 RNAi#29 | 2.86 | 7.388 |

As shown in Tables 16 and 17 and FIG. 12 to FIG. 16, compared with wild-type WT, the MfERF026 transgenic line with inhibited expression had higher survival rate, better cell membrane permeability, lower damage, and obviously stronger drought tolerance.

Before and after the experiment, the physiological and biochemical indicators of M. sativa in the experimental group and the control group were detected using kits from Suzhou Comin Biotechnology Co., Ltd.

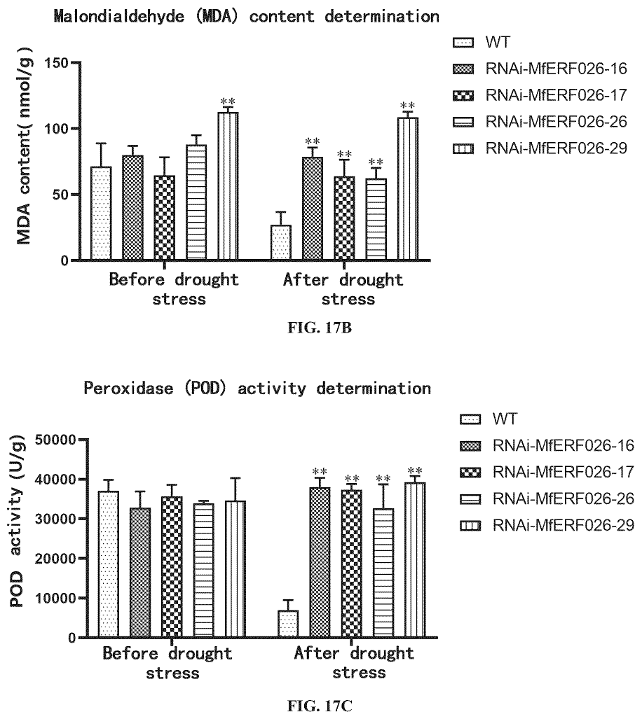

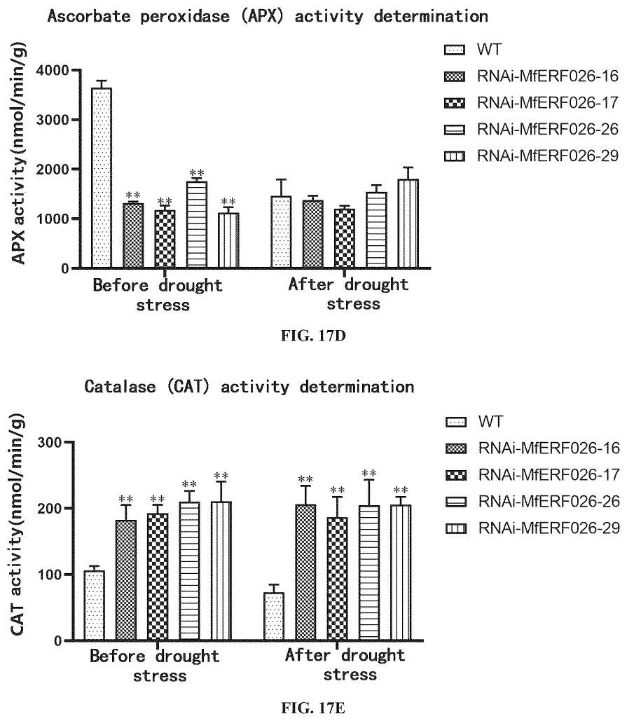

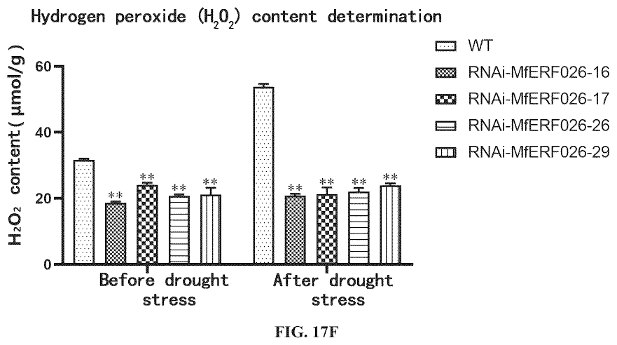

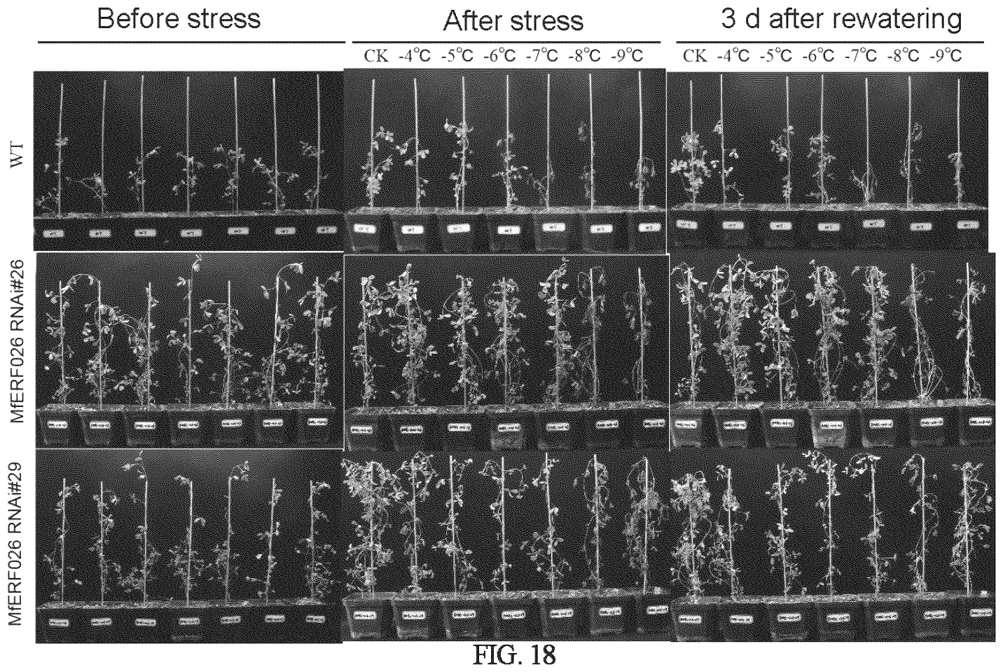

The measurement results of physiological and biochemical indicators of wild-type M. sativa and transgenic M. saliva under drought stress are shown in Table 18 and FIGS. 17A-17F.

TABLE 18
Measurement results of physiological and biochemical indicators of wild-type M. sativa and
transgenic M. sativa under drought stress

| | APX | | CAT | | H₂O₂ | | MDA | | POD | | SOD | |
|---|---|---|---|---|---|---|---|---|---|---|---|---|
| Group | Before | After | Before | After | Before | After | Before | After | Before | After | Before | After |
| WT | 3648 | 1467 | 106.7 | 73.44 | 31.67 | 53.86 | 71.45 | 27.32 | 37151 | 6983 | 255.5 | 110.9 |
| #16 | 1320 | 1382 | 183 | 206.3 | 18.72 | 20.91 | 79.95 | 78.79 | 32868 | 38041 | 202.2 | 321.1 |
| #17 | 1180 | 1207 | 192.8 | 187.1 | 24.11 | 21.3 | 64.8 | 64.09 | 35717 | 37394 | 273.6 | 287.6 |
| #26 | 1761 | 1549 | 210.7 | 205.2 | 20.76 | 22.14 | 88 | 62.51 | 33983 | 32628 | 290 | 287.6 |
| #29 | 1126 | 1803 | 210.8 | 206.2 | 21.18 | 24.01 | 112.9 | 108.7 | 34745 | 39326 | 290.5 | 317.2 |

Note:
in the table, “before” represented before stress, and “after” represented after stress.

As shown in Table 18 and FIGS. 17A-17F, interfering with MfERF026 enhanced the antioxidant capacity of the antioxidant enzyme system of transgenic M. sativa, including increasing the activities of SOD and POD and increasing the content of CAT, thereby making the H₂O₂ content significantly lower than that of the wild-type before and after stress.

(4) Cold Tolerance Test of Transgenic Lines with Inhibited Expression

The wild-type plants and transgenic lines #26 and #29 grown for 30 d in a soil matrix with vermiculite and nutrient soil at a volume ratio of 3:2 were treated in a low-temperature incubator, where the incubation program was: 0° C. 1 h, −1° C. 1 h, −2° C. 1 h, −3° C. 1 h, −4° C. 1 h, −5° C. 1 h, −6° C. 1 h, −7° C. 1 h, −8° C. 1 h, −9° C. 1 h, and then cultured overnight at 4° C. After −4° C. 1 h, some plants were taken out and immediately placed in a 4° C. plant incubator for overnight cultivation. Every time the temperature dropped by one degree centigrade, part of the plant material was taken out and immediately placed in a 4° C. plant incubator for overnight cultivation. The next day, all plant materials were placed at room temperature and recovered for 3 d. After 3 d of recovery, the survival rates of M. sativa in the experimental group and the control group were calculated. The changes in electric conductivity of M. sativa in the experimental group and the control group were detected and compared before and after the experiment. The CK control group was cultured at room temperature under the same experimental conditions. The calculation method of the electric conductivity was the same as that in (3).

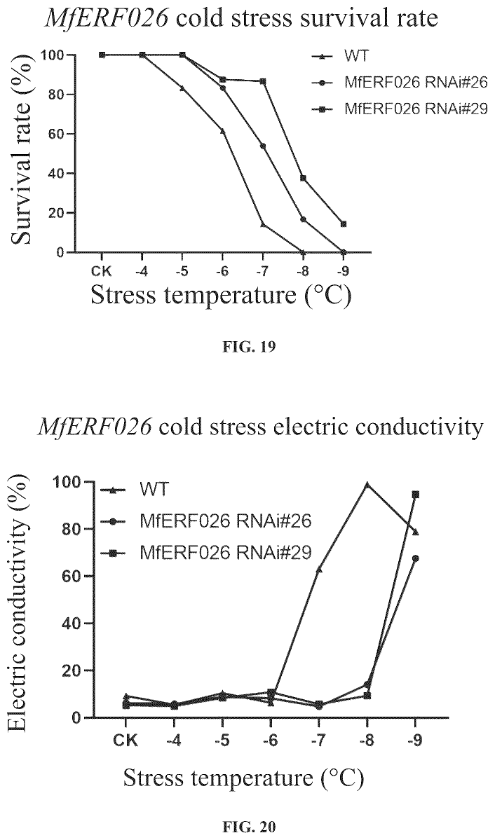

FIG. 18 shows the growth of plants during the cold tolerance test of the wild-type and transgenic lines. Table 19 and FIG. 19 show the survival rates of the cold tolerance test of wild-type and transgenic lines. Table 20 and FIG. 20 show the detection results of the electrical conductivity of the cold tolerance test of the wild-type and transgenic lines.

TABLE 19
Survival rates of cold tolerance test of wild-type and transgenic lines

| Survival rate | CK | −4 | −5 | −6 | −7 | −8 | −9 |
|---|---|---|---|---|---|---|---|
| WT | 100% | 100% | 83.33% | 61.54% | 14.29% | 0% | 0% |
| MfERF026 | 100% | 100% | 100% | 83.33% | 53.85% | 16.67% | 0% |
| RNAi#26 | | | | | | | |
| MfERF026 | 100% | 100% | 100% | 87.50% | 86.67% | 37.50% | 14.29% |
| RNAi#29 | | | | | | | |

TABLE 20
Detection results of electrical conductivity of cold tolerance test of wild-type and transgenic lines

| Survival rate | CK | −4 | −5 | −6 | −7 | −8 | −9 |
|---|---|---|---|---|---|---|---|
| WT | 9.23% | 5.50% | 10.34% | 6.31% | 63% | 98.91% | 78.91% |
| MfERF026 | 6.14% | 5.79% | 8.83% | 8.17% | 4.72% | 14.00% | 67.51% |
| RNAi#26 | | | | | | | |
| MfERF026 | 5.25% | 4.99% | 8.41% | 10.82% | 5.74% | 9.32% | 94.65% |
| RNAi#29 | | | | | | | |

As shown in Tables 19 and 20 and FIG. 18 and FIG. 19, compared with wild-type WT, the MfERF026 transgenic line with inhibited expression had a higher survival rate under cold stress at −5° C. to −8° C. As shown in Table 20 and FIG. 20, the MfERF026 transgenic line with inhibited expression had a lower electrical conductivity, more complete cell membrane permeability, and lower degree of damage under cold stress at −7° C. and −8° C. These results indicated that transgenic plants showed better cold tolerance after inhibited expression of MfERF026.

In summary, the inhibited expression of the ERF MfERF026 in M. falcata provided by the present disclosure in the M. sativa may increase the drought tolerance and cold tolerance of M. sativa, and promote the growth and development of M. sativa, thereby increasing the growth rate and/or increasing the number of leaves for M. sativa.

Although the above examples have described the present disclosure in detail, it is only a part of, not all of the embodiments of the present disclosure. Other embodiments may also be obtained by persons based on the example without creative efforts, and all of these embodiments shall fall within the protection scope of the present disclosure.

What is claimed is:

1. A method for promoting growth and development of Medicago sativa (M. sativa), comprising contacting the M. sativa with a regulatory agent for MfERF026, wherein MfERF026 encodes a protein having the amino acid sequence of SEQ ID NO: 2.

2. The method according to claim 1, wherein MfERF026 has the nucleotide sequence of SEQ ID NO: 1.

3. The method according to claim 1, wherein the regulatory agent comprises an agent that silences or reduces expression of MfERF026.

4. The method according to claim 3, wherein the agent that silences or reduces the expression of MfERF026 comprises an interfering RNA for the expression of MfERF026.

5. A trans-acting small interfering RNA (tasiRNA), wherein the tasiRNA has the nucleotide sequence of SEQ ID NO: 18.

6. A base fragment for reducing MfERF026 expression obtained based on the tasiRNA according to claim 5, wherein the base fragment has the nucleotide sequence of SEQ ID NO: 17.

7. A primer pair for amplifying the base fragment according to claim 6, wherein the primer pair comprises an upstream primer MfERF026 RNAi-F and a downstream primer MfERF026 RNAi-R; the upstream primer MfERF026 RNAi-F having the nucleotide sequence of SEQ ID NO: 14; and the downstream primer MfERF026 RNAi-R having the nucleotide sequence of SEQ ID NO: 15.

8. An expression vector for reducing MfERF026 expression, wherein the expression vector comprises the base fragment according to claim 6.

9. An engineering bacterium constructed based on the expression vector according to claim 8.

10. A method for constructing transgenic M. sativa, comprising the following steps:
infecting a M. sativa leaf with the engineering bacterium according to claim 9 to obtain a transformed M. sativa leaf; and
subjecting the transformed M. sativa leaf to tissue culture to obtain the transgenic M. sativa with inhibited MfERF026 expression.
